(19) United States
(12) Patent Application Publication
(10) Pub. No.: US 2003/0072744 A1
(43) Pub. Date: Apr. 17, 2003

(54) Methods of altering cardiac cell phenotype

(75) Inventor: Robert L. Engler, San Diego, CA (US)

Correspondence Address: GARY CARY WARE & FRIENDENRICH LLP, 4365 EXECUTIVE DRIVE, SUITE 1600, SAN DIEGO, CA, 92121-2189, US

(73) Assignee: COLLATERAL THERAPEUTICS

(21) Appl. No.: 10/247,274

(22) Filed: Sep. 18, 2002

Related U.S. Application Data
(63) Continuation of application No. 09/745,185, filed on Dec. 19, 2000.
Application No. 09/745,185 is a continuation of application No. 09/322,400, filed on May 28, 1999, now abandoned.
Application No. 09/745,185 is a continuation of application No. PCT/US99/11961, filed on May 28, 1999.
(60) Provisional application No. 60/087,380, filed on May 30, 1998.

(51) Int. Cl. A61K 48/00

(52) U.S. Cl. 424/93.21; 514/44

(57) ABSTRACT

Methods for improving or maintaining cardiac function in patients are disclosed. The methods include the stimulation of heart muscle regeneration, the treatment of patients with congestive heart failure and the prevention of organ transplant rejection. Methods are also disclosed for the treatment of patients after myocardial infarction and/or patients with congestive heart failure by adenovirus-mediated delivery of peptides, including, but not limited to, NKX-2.5, MEF2, GATA4, BCL-2, HGH, and Fas ligand, that alter the phenotype of cells in the heart.

This application claims priority to Provisional Application Engler, U.S. Serial No. 60/087,380, filed May 30, 1998, entitled METHODS OF ALTERING THE PHENOTYPIC EXPRESSION OF CARDIAC CELLS TO IMPROVE OR MAINTAIN CARDIAC FUNCTION.

### FIELD OF THE INVENTION

The present invention relates to methods for improving or maintaining cardiac function in patients. More particularly, the invention relates to methods for altering the phenotype of cells in the heart to improve or maintain cardiac function using genes delivered to the heart. The invention also relates to methods of using gene therapy to produce hormones to improve or maintain cardiac function.

### BACKGROUND OF THE INVENTION

Heart Muscle Necrosis and Myocardial Infarction

Atherosclerosis of the coronary arteries which supply the heart with blood often leads to acute thrombotic coronary occlusion and myocardial cell death and “infarction.” Cardiomyocytes, the heart muscle cells, are terminally differentiated cells that are generally incapable of cell division. When they die during the course of acute myocardial infarction they are generally replaced by scar tissue. A typical process is as follows. In the first few days after acute infarction, cardiomyocytes lose membrane integrity and attract polymorphonuclear leukocytes (PMNs) that help digest the necrotic cells. These cardiomyocytes also may die due to apoptosis or programmed cell death. This PMN phase generally lasts about 3 days and is followed by invasion of lymphocytes and macrophages that finish clearing the dead or necrotic cells and orchestrate the proliferation of fibrocytes into myofibroblasts. Myofibroblast proliferation and production of collagen characterize the granulation phase of healing, beginning several days after infarction and lasting for 2 or 3 weeks. Finally a collagen rich scar is formed. Scar tissue is not contractile, fails to contribute to cardiac function, and often plays a detrimental role by expanding during cardiac contraction, or by increasing the size and effective radius of the ventricle. The larger the ventricle, the greater the wall stress, thus the more force the remaining muscle cells must generate in order to pump blood. A method of replacing the lost cardiomyocytes is needed in order to mitigate the effect of this muscle necrosis and thus improve cardiac function after myocardial infarction.

There have been suggestions regarding cell therapies and other approaches for potentially treating heart conditions such as those associated with myocardial infarct. See, e.g., Yoon, P., et al., Tex Heart Inst J (1995) 22:119-125; Soonpaa, M., et al., Ann NY Acad Sci (1995) 752:446-454; Murry, C. E. et al., J Clin Invest (1996) 98:2209-17; Li, R.-K. et al., Can J Cardiol (1998) 14:735-744; and see Leor, J., et al., Cardiovasc Res (1997) 35:431-441 and references reviewed therein. For example, R. Kloner and colleagues suggested delivering skeletal muscle differentiation genes such as MyoD to cardiac tissue. See, e.g., WO95/12979, published May 18, 1995. Subsequent scientific publications regarding the potential use of MyoD in animal infarct models reported some delivery of the gene which appeared to induce limited differentiation of the skeletal muscle type. See, e.g., Murry, C. E. et al., supra, and Leor, J. et al., supra. These approaches do not appear capable of generating functioning cardiac myocytes. Cell transplant techniques might be used to provide cardiac myocytes but such procedures have been limited by technical hurdles including limitations of suitable cell sources, difficulties associated with cell expansion and viability, and potential rejection of grafted cells. See, e.g., the articles and reviews cited above.

Congestive Heart Failure

It has been reported that 3-4 million adults in the United States have congestive heart failure (abbreviated “CHF” herein); and the incidence of CHF is increasing (see, e.g., Baughman, K., Cardiology Clinics 13: 27-34, 1995). CHF is defined as abnormal heart function resulting in inadequate cardiac output for metabolic needs (Braunwald, E. (ed), In: Heart Disease, W. B. Saunders, Philadelphia, page 426, 1988). Symptoms include breathlessness, fatigue, weakness, leg swelling, and exercise intolerance. On physical examination, patients with heart failure tend to have elevations in heart and respiratory rates, rales (an indication of fluid in the lungs), edema, jugular venous distension, and, in general, enlarged hearts. The most common cause of CHF is atherosclerosis which causes blockages in the blood vessels (coronary arteries) that provide blood flow to the heart muscle. Ultimately such blockages may cause myocardial infarction (death of heart muscle) with subsequent decline in heart function and resultant heart failure. Other causes of CHF include valvular heart disease, idiopathic or familial dilated cardiomyopathy hypertension, viral infections of the heart, alcohol, and diabetes. Some cases of heart failure occur without clear etiology and are called idiopathic.

Annually in U.S. hospitals, CHF is the most frequent non-elective admission, and is the discharge diagnosis for approximately 500,000 patients. Once symptoms of heart failure are moderately severe, the prognosis is worse than most cancers in that 50% of such patients may die within 2 years (Braunwald, E. (ed), In: Heart Disease, W. B. Saunders, Philadelphia, page 471-485, 1988). Although medical therapy can initially attenuate the symptoms of heart failure (e.g., edema, breathlessness and fluid in the lungs), and in some cases prolong life, the prognosis for this disease, even with medical treatment, is grim (see, e.g., Baughman, K., Cardiology Clinics 13: 27-34, 1995).

Present treatments for CHF include pharmacological therapies, coronary revascularization procedures (e.g. coronary artery bypass surgery and angioplasty), and heart transplantation. Pharmacological therapies have been directed toward increasing the force of contraction of the heart (by using inotropic agents such as digitalis and β-adrenergic receptor agonists), reducing fluid accumulation in the lungs and elsewhere (by using diuretics), and reducing the work of the heart (by using agents that decrease systemic vascular resistance such as angiotensin converting enzyme or “ACE” inhibitors).

β-adrenergic receptor antagonists have also been tested. While such pharmacological agents can improve symptoms, and potentially prolong life, the prognosis in most cases remains dismal.

Methods for treating congestive heart failure by delivery of genes to the heart have been described by Hammond et al. in PCT Publication WO98/10085, published Mar. 12, 1998, hereby incorporated by reference. These methods involve the delivery of genes encoding elements of the β-adrenergic-receptor signaling pathway to the heart of a patient with a heart disease. Methods for promoting angiogenesis in the heart, which can also be used for preventing and/or treating CHF, have been described by Hammond et al. in PCT Publication WO96/26742, published Sep. 6, 1996; U.S. Pat. No. 5,792,453, issued Aug. 11, 1998; and, PCT Publication WO98/50079, published Apr. 30, 1998, all of which are incorporated by reference herein.

During chronic congestive heart failure (CHF) there is progressive dilation of the heart and worsening of heart function. Human growth hormone (HGH) is a circulating peptide hormone that has shown some promise as a therapeutic agent for CHF. However, peptides must be given by repeated injection, and systemic effects were induced by this multifunctional hormone. For example, high levels of growth hormone present systemically can induce glucose intolerance and diabetes.

Chronic CHF is also associated with the progressive loss of cardiomyocytes by apoptosis, as has been observed in animal models. Apoptosis, or programmed cell death, is a tightly regulated process. Specific intracellular proteins are believed to provide blocks for apoptosis in a concentration-dependent fashion.

Rejection of Transplanted Organs

The T-cell-mediated immune response is the primary mechanism of acute and chronic rejection of organ transplants. Restricted areas of the body such as the lens of the eye enjoy relative immune “privilege” in that they seem to be protected from immune attack by T lymphocytes even though they may contain foreign antigens. Recently, it was suggested that the source of the immune privilege was the display of Fas ligand. Fas ligand is a cell surface receptor displayed or induced by external signals on a variety of cell types, but most notably on immune-competent lymphocytes. Fas signaling is the mechanism whereby self-reactive clones (lymphocytes that recognize self antigens) are deleted, in order to prevent immunity to the organism's own cells. Fas signaling results in programmed cell death, or apoptosis. Engagement of the Fas receptor by Fas ligand results in activation of apoptosis. The immune system uses display of Fas ligand to induce death of cells displaying Fas receptor. Thus, areas of immune privilege have been found to display Fas ligand and thus induce a programmed cell death response in lymphocytes. See, e.g., Lau, H. T. et al., Science 273:109-112 (1996); and Griffith, T. S. et al., Science, 270: 1189-1192 (1995).

The invention described and claimed herein addresses and overcomes problems and limitations associated with the prior art methods of treating heart diseases such as congestive heart failure, myocardial infarction, and organ transplant.

### SUMMARY OF THE INVENTION

The present invention is directed to methods for improving or maintaining cardiac function in a patient using gene therapy.

It is a novel aspect of this invention that heart muscle regeneration may be stimulated, preferably after myocardial infarction. Preferably, such heart muscle regeneration will mitigate the effect of muscle necrosis and improve heart performance. In preferred embodiments, such methods involve the delivery of genes to cells in the heart.

It is another novel aspect of this invention that congestive heart failure may be alleviated, and cardiac function thereby enhanced by the delivery of a human growth hormone gene to heart cells.

It is another novel aspect of this invention that congestive heart failure may be alleviated and cardiac function thereby enhanced by reducing cell loss in the failing heart by the delivery of a gene coding for BCL-2, or other anti-apoptotic proteins.

It is another novel aspect of this invention that organ rejection after transplantation, and/or atherosclerosis, may be prevented or alleviated by expression of Fas ligand on the plasma membrane of cells in transplanted organs.

In the methods of the invention, the nucleic acid coding for the cardiac function-enhancing peptide such as, for example, NKX-2.5, MEF2, GATA4, BCL-2, FAS ligand, or HGH, or other cardiomyocyte differentiating peptide, anti-apoptotic protein, or growth hormone, is preferably in a recombinant vector. This recombinant vector is preferably delivered to cells in the heart. Preferably, the vector is introduced into a blood vessel supplying blood to the myocardium of the heart, so as to deliver the vector to cells within the heart; more preferably the vector is introduced into the lumen of a coronary artery, a saphenous vein graft, or an internal mammary artery graft. Most preferably, the vector is introduced into the lumen of one or more coronary arteries. In preferred aspects of the invention, the vector is a viral vector, preferably a replication-deficient adenovirus vector or an adeno-associated virus (AAV) vector. Preferably, the nucleic acid coding for the cardiac function-enhancing peptide(s) such as, for example, NKX-2.5, MEF2, GATA4, BCL-2, or HGH, or other cardiomyocyte differentiating peptide, anti-apoptotic protein, or growth hormone, is operably linked to a promoter sequence that is active in the target cell. Preferably, the promoter is a heart cell-specific promoter. Where, for example, expression is desired in cardiomyocytes, the alpha-myosin heavy chain promoter may be used.

In one aspect of the present invention, a cardiomyocyte differentiating peptide such as NKX-2.5 (or another NKX factor such as NKX-2.3, NKX-2.6 and the like), MEF2 (including the various homologs thereof such as MEF2 A, B, C or D), or GATA4 (or another GATA factor such as GATA5, GATA6 and the like), or a combination comprising one or more of such peptides, is used to induce the conversion of cells associated with a myocardial infarction or scar (such as fibroblasts), into functional cardiac myocytes which can contract and thus contribute to the function of the myocardium.

In preferred aspects, the replication-deficient adenovirus or AAV vector is delivered to the myocardium of the patient by intracoronary injection directly into one or more coronary arteries, said vector comprising transgene(s) coding for one or more peptides selected from the group consisting of NKX-2.5, MEF2, GATA4, BCL-2, and HGH, or other cardiomyocyte differentiating peptide, anti-apoptotic protein, or growth hormone, and capable of expressing the transgene in the myocardium. Preferably, the patient is human. The patient may exhibit congestive heart failure, myocardial infarction and/or other heart disease.

In preferred aspects, a single injection of said vector is delivered. Preferably, about 10¹⁰ to about 10¹⁴ adenovirus vector particles are delivered in the injection. More preferably, about 10¹¹ to about 10¹³ adenovirus vector particles are delivered in the injection. Most preferably, about 10¹² adenovirus vector particles are delivered in the injection. In preferred aspects, the transgene is driven by a CMV promoter which is contained in the vector. In other preferred aspects, the transgene is driven by a heart cell-specific promoter which is contained in the vector. Exemplary heart cell-specific promoters include a ventricular myosin light chain-2 promoter and an alpha myosin heavy chain promoter.

In preferred aspects, the intracoronary injection is conducted about 1-3 cm into the lumens of coronary arteries. In further aspects of these methods of the invention, the patient may be treated with a stent.

Also provided as an aspect of the invention is an injectable adenovirus vector preparation, comprising a recombinant adenoviral vector, said vector containing little or no wild-type virus and comprising: a partial adenoviral sequence from which at least the E1A and E1B genes, the E1A, E1B and E4 genes, the E1A, E1B and E3 genes, or the E1A, E1B and E2 genes have been inactivated or deleted, and a transgene coding for a cardiac function-enhancing peptide such as, for example, NKX-2.5, MEF2, GATA4, BCL-2, or HGH, or other cardiomyocyte differentiating peptide, anti-apoptotic protein, or growth hormone, driven by a promoter operably linked to the transgene; and a pharmaceutically acceptable carrier.

For purposes of the present invention, the term “patient” refers to any mammal, preferably humans. According to the preceding methods of the claimed invention, a single injection of said vector may be delivered. Preferably, about 10¹⁰ to about 10¹⁴, more preferably about 10¹¹ to about 10¹³, and most preferably about 10¹² adenovirus vector particles are delivered in the injection. Those skilled in the art will routinely optimize the level by dose-response experiments using well-known animal models.

In preferred methods of the invention, the vector comprises a transgene coding for the cardiac function-enhancing peptide, such as, for example, NKX-2.5, MEF2, GATA4, BCL-2, or HGH, or other cardiomyocyte differentiating peptide, anti-apoptotic protein, or growth hormone, operably linked to, or driven by, a heterologous constitutive promoter or a heterologous inducible promoter. A preferred heterologous constitutive promoter is a CMV promoter. The CMV promoter may also include an enhancer. In other preferred embodiments described herein, the promoter is a tissue-specific promoter, preferably a cardiac-specific promoter, more preferably, a cardiomyocyte-specific promoter. Preferred examples of cardiomyocyte-specific promoters include an alpha-myosin heavy chain promoter, a ventricular myosin light chain 2 promoter and a ventricular myosin heavy chain promoter. The transgene may also be operably linked to a heterologous enhancer, such as the CMV enhancer. Preferably, the transgene is operably linked to a polyadenylation signal. The vector may also comprise a detectable marker gene, a selectable marker gene, and/or an enhancer.

In preferred aspects, the intracoronary injection is conducted about 1-3 cm into the lumens of one or more coronary arteries. In other preferred aspects, the intracoronary injection is conducted about 1-3 cm into the lumens of a saphenous vein graft and/or an internal mammary artery graft in addition to coronary artery.

The cardiac tissue supplied with blood by the coronary arteries can also be reached by coronary sinus retroperfusion, as has been used for drug therapy. (See, e.g., Gore, J., et al., Circ. 74:381 (1986); Meerbaum, S., et al. J. Am. Coll. Cardiol. 1:1262-7 (1983); Povzhitkov, M., J. Am. Coll. Cardiol. 3:939-47 (1984)).

In preferred methods of enhancing cardiac function according to one of the preceding embodiments, the vector is a viral vector or a lipid-based vector, preferably a viral vector. The vector can be a targeted vector, especially a targeted vector that preferentially binds to ventricular myocytes, endothelial cells, or fibroblasts. Presently preferred viral vectors are derived from adenovirus (Ad) or adeno-associated virus (AAV). Both human and non-human viral vectors can be used but preferably the recombinant viral vector is replication-defective in humans. Where the vector is an adenovirus, it preferably comprises a polynucleotide having a promoter operably linked to a gene encoding a cardiac function-enhancing protein or peptide, and is replication-defective in humans.

Presently preferred replication-defective adenoviral vectors have been changed to inactivate or delete the E1A and E1B genes, the E1A, E1B and E4 genes, the E1A, E1B and E3 genes, or the E1A, E1B and E2 genes. Preferably about 10¹⁰ to 10¹⁴ adenovirus vector particles, more preferably about 10¹¹ to 10¹³ vector particles, most preferably about 10¹² vector particles, are introduced into a blood vessel, preferably a blood vessel supplying the myocardium.

For AAV vectors, the vector preferably comprises a polynucleotide having a promoter operably linked to a gene encoding a cardiac function-enhancing protein or peptide, and preferably, the gene encoding the protein or peptide is flanked by AAV inverted terminal repeats (ITRs). Preferably, the AAV vector is replication-defective in humans. Presently preferred replication-defective AAV vectors have deletions affecting one or more AAV replication or encapsidation sequences. Alternatively, the vector can be a lipid-based vector comprising a gene encoding a cardiac function-enhancing protein or peptide as described herein.

Thus, in preferred embodiments, the present invention provides a method for improving or maintaining cardiac function in a patient comprising delivering a vector to the heart of said patient, said vector comprising a transgene coding for a cardiomyocyte differentiating peptide. The cardiomyocyte differentiating peptide is preferably selected from the group consisting of NKX-2.5 (or other NK factor), MEF2 (including various homologs thereof), and GATA4 (or other GATA factor). In other aspects, the present invention provides a method for stimulating heart muscle regeneration (e.g., after myocardial infarction) in a patient comprising delivering a vector comprising a transgene to the heart of said patient, wherein said transgene encodes a cardiomyocyte-differentiating peptide. Preferably, the cardiomyocyte-differentiating peptide is selected from the group consisting of NKX-2.5, MEF2, and GATA4. In other preferred embodiments, the present invention provides a method for improving or maintaining cardiac function in a patient comprising delivering a vector to the heart of said patient, said vector comprising a transgene coding for BCL-2 or other anti-apoptotic protein. In other aspects, the present invention provides a method for treating congestive heart failure in a patient comprising delivering a vector comprising a transgene coding for BCL-2 or other apoptotic protein to the heart of said patient. Preferably, this treatment prevents cardiac cell loss. More preferably, this treatment renders the cardiomyocytes more resistant to apoptosis.

In other aspects, the invention provides a method for improving or maintaining cardiac function in a patient comprising delivering a vector comprising a transgene coding for HGH to the heart of said patient. In other preferred aspects, the HGH transgene is fused at its 5′ end to a proteoglycan binding domain of VEGF145. In all of the above aspects, the vector is preferably delivered to cardiac myocytes. Preferably, the transgene is a human gene. In preferred methods, the vector is delivered by intracoronary injection into one or both coronary arteries. Preferably, the vector is delivered to a blood vessel supplying blood to the myocardium of the heart, wherein said blood vessel is selected from the group consisting of a coronary artery, a saphenous vein graft, and an internal mammary artery graft. Preferably the vector is a replication-deficient adenovirus vector.

In yet other preferred embodiments, a kit is provided for intracoronary injection of a recombinant vector expressing a peptide selected from the group consisting of NKX-2.5, MEF2, GATA4, BCL-2, HGH, and FAS ligand or other cardiomyocyte differentiating peptide, anti-apoptotic protein, or growth hormone, comprising: a nucleic acid molecule encoding NKX-2.5, MEF2, GATA4, BCL-2, HGH, or Fas ligand or other cardiomyocyte differentiating peptide, anti-apoptotic protein, or growth hormone, cloned into a vector suitable for expression of said polynucleotide in a heart cell, a suitable container for said vector, and instructions for injecting said vector into a patient. Preferably, the polynucleotide is cloned into an adenovirus expression vector. In other preferred aspects, an inflatable balloon catheter coated with said vector is employed to deliver said transgene. In yet other preferred aspects, a filtered injectable adenovirus vector preparation is provided comprising: a recombinant adenoviral vector, said vector containing no wild-type virus and comprising: a partial adenoviral sequence from which the E1A and E1B genes, the E1A, E1B and E4 genes, the E1A, E1B and E3 genes, or the E1A, E1B and E2 genes have been inactivated or deleted, and a transgene coding for a NKX-2.5, MEF2, GATA4, BCL-2, HGH, or Fas ligand or other cardiomyocyte differentiating peptide, anti-apoptotic protein, or growth hormone, driven by a promoter flanked by the partial adenoviral sequence; and a pharmaceutically acceptable carrier.

In another preferred embodiment, the invention provides a method of reducing the likelihood of rejection of a transplanted organ in a patient comprising delivering a vector to the transplanted organ, said vector comprising a transgene coding for a Fas ligand. Preferably, the transplanted organ is heart tissue including, preferably, the entire heart. In one preferred aspect, the vector is delivered to the transplanted organ before transplanting the organ into a patient. Preferably, the vector is delivered in the organ preservation solution used to preserve the organ prior to transplantation. In one preferred method, the vector is in a solution, such as an organ preservation solution. This solution may be pumped through the organ using a perfusion device. Preferably, the organ is washed after perfusion, prior to transplantation. In another preferred aspect, the vector is delivered to the transplanted organ after transplanting the organ into a patient. Preferably, the patient is human. The transgene may be delivered according to methods known to those of ordinary skill in the art. Preferably, the transgene is delivered using the vectors and delivery methods of the present application.

### DETAILED DESCRIPTION OF PREFERRED EMBODIMENTS

A “polynucleotide” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified polynucleotides such as methylated and/or capped polynucleotides.

“Recombinant,” as applied to a polynucleotide, means that the polynucleotide is the product of various combinations of cloning, restriction and/or ligation steps, and other procedures that result in a construct that is distinct from a polynucleotide found in nature.

A “gene” refers to a polynucleotide or portion of a polynucleotide comprising a sequence that encodes a protein. For most situations, it is desirable for the gene to also comprise a promoter operably linked to the coding sequence in order to effectively promote transcription. Enhancers, repressors and other regulatory sequences may also be included in order to modulate activity of the gene, as is well known in the art. (See, e.g., the references cited below).

The terms “polypeptide,” “peptide,” and “protein” are used interchangeably to refer to polymers of amino acids of any length. These terms also include proteins that are post-translationally modified through reactions that include glycosylation, acetylation and phosphorylation.

The term “HGH” refers to human growth hormone or a derivative thereof which retains the stimulatory activity of human growth hormone. Preferred derivatives have activities that are within 10% to 1,000% of the activity, more preferably within 50% to 200%.

A “cardiac function enhancing peptide” is a peptide that improves or maintains cardiac function when expressed in a heart cell, or when present in the environment surrounding the heart cell.

A “cardiomyocyte differentiating peptide” is a protein or factor that is involved in the differentiation of cells along the cardiomyocyte lineage. These include, for example, various hormones, factors, and cell determinants, such as NKX-2.5, MEF2, and GATA4 and similar differentiation factors.

“Derivatives” of a cardiac-function enhancing peptide such as, for example, NKX-2.5, MEF2, GATA4, Fas ligand, BCL-2, or HGH, or other cardiomyocyte differentiating peptide, anti-apoptotic protein, or growth hormone, are functional equivalents having similar amino acid sequences and retaining, to some extent, the activities of the cardiac-function enhancing peptide. By “functional equivalent” is meant the derivative has an activity that can be substituted for the activity of the cardiac-function enhancing peptide. Preferred functional equivalents retain the full level of activity of the cardiac-function enhancing peptide as measured by assays known to those skilled in the art, and/or in the assays described herein. Preferred functional equivalents have activities that are within 10% to 1,000% of the activity of the cardiac-function enhancing peptide, more preferably within 50% to 200%. Derivatives have at least 50% sequence similarity, preferably 70%, more preferably 90%, and even more preferably 95% sequence similarity to the cardiac-function enhancing peptide. “Sequence similarity” refers to “homology” observed between amino acid sequences in two different polypeptides, irrespective of polypeptide origin.

The ability of the derivative to retain some activity can be measured using techniques described herein and/or using techniques known to those skilled in the art for measuring the activity of the cardiac-function enhancing peptide. Derivatives include modification occurring during or after translation, for example, by phosphorylation, glycosylation, crosslinking, acylation, proteolytic cleavage, linkage to an antibody molecule, membrane molecule or other ligand (see Ferguson et al., 1988, Annu. Rev. Biochem. 57:285-320).

Specific types of derivatives also include amino acid alterations such as deletions, substitutions, additions, and amino acid modifications. A “deletion” refers to the absence of one or more amino acid residue(s) in the related polypeptide. An “addition” refers to the presence of one or more amino acid residue(s) in the related polypeptide. Additions and deletions to a polypeptide may be at the amino terminus, the carboxy terminus, and/or internal. Amino acid “modification” refers to the alteration of a naturally occurring amino acid to produce a non-naturally occurring amino acid. A “substitution” refers to the replacement of one or more amino acid residue(s) by another amino acid residue(s) in the polypeptide. Derivatives can contain different combinations of alterations including more than one alteration and different types of alterations.

Although the effect of an amino acid change varies depending upon factors such as phosphorylation, glycosylation, intra-chain linkages, tertiary structure, and the role of the amino acid in the active site or a possible allosteric site, it is generally preferred that the substituted amino acid is from the same group as the amino acid being replaced. To some extent the following groups contain amino acids which are interchangeable: the basic amino acids lysine, arginine, and histidine; the acidic amino acids aspartic and glutamic acids; the neutral polar amino acids serine, threonine, cysteine, glutamine, asparagine and, to a lesser extent, methionine; the nonpolar aliphatic amino acids glycine, alanine, valine, isoleucine, and leucine (however, because of size, glycine and alanine are more closely related and valine, isoleucine and leucine are more closely related); and the aromatic amino acids phenylalanine, tryptophan, and tyrosine. In addition, although classified in different categories, alanine, glycine, and serine seem to be interchangeable to some extent, and cysteine additionally fits into this group, or may be classified with the polar neutral amino acids.

Although proline is a nonpolar neutral amino acid, its replacement represents difficulties because of its effects on conformation. Thus, substitutions by or for proline are not preferred, except when the same or similar conformational results can be obtained. The conformation conferring properties of proline residues may be obtained if one or more of these is substituted by hydroxyproline (Hyp).

Examples of modified amino acids include the following: altered neutral nonpolar amino acids such as amino acids of the formula H₂N(CH₂)ₙCOOH where n is 2-6, sarcosine (Sar), t-butylalanine (t-BuAla), t-butylglycine (t-BuGly), N-methyl isoleucine (N-MeIle), and norleucine (Nleu); altered neutral aromatic amino acids such as phenylglycine; altered polar, but neutral amino acids such as citrulline (Cit) and methionine sulfoxide (MSO); altered neutral and nonpolar amino acids such as cyclohexyl alanine (Cha), altered acidic amino acids such as cysteic acid (Cya); and altered basic amino acids such as ornithine (Orn).

Preferred derivatives have one or more amino acid alteration(s) that do not significantly affect the receptor-binding or other activity of the cardiac-function enhancing peptide. In regions of the cardiac-function enhancing peptide sequence not necessary for the cardiac-function enhancing peptide activity, amino acids may be deleted, added or substituted with less risk of affecting activity. In regions required for the cardiac-function enhancing peptide, amino acid alterations are less preferred as there is a greater risk of affecting activity. Such alterations should be conservative alterations. For example, one or more amino acid residues within the sequence can be substituted by another amino acid of a similar polarity which acts as a functional equivalent.

Conserved regions tend to be more important for protein activity than non-conserved regions. Standard procedures can be used to determine the conserved and non-conserved regions important for activity using in vitro mutagenesis techniques or deletion analyses and to measure activity as described by the present disclosure.

Derivatives can be produced using standard chemical techniques and recombinant nucleic acid molecule techniques. Modifications to a specific polypeptide may be deliberate, as through site-directed mutagenesis and amino acid substitution during solid-phase synthesis, or may be accidental such as through mutations in hosts which produce the polypeptide. Polypeptides including derivatives can be obtained using standard techniques such as those described in Sambrook et al., Molecular Cloning, Cold Spring Harbor Laboratory Press (1989). For example, Chapter 15 of Sambrook describes procedures for site-directed mutagenesis of cloned DNA.

In one aspect the invention features a nucleic acid molecule, or polynucleotide encoding a cardiac-function enhancing peptide. In some situations it is desirable for such nucleic acid molecule to be isolated or enriched, or purified. By the use of the term “enriched” in reference to nucleic acid molecule, polypeptide, or protein is meant that the specific DNA or RNA sequence, polypeptide, or protein constitutes a significantly higher fraction (2-5 fold) of the total DNA, RNA, polypeptide or protein present in the cells or solution of interest than in normal or diseased cells or in the cells from which the sequence was taken. This could be caused, for example, by a person by preferential reduction in the amount of other DNA or RNA present, or by a preferential increase in the amount of the specific DNA or RNA sequence, or by a combination of the two. However, it should be noted that enriched does not imply that there are no other DNA or RNA sequences present, just that the relative amount of the sequence of interest has been significantly increased. The term significant here is used to indicate that the level of increase is useful to the person making such an increase, and generally means an increase relative to other nucleic acids of about at least 2 fold, more preferably at least 5 to 10 fold or even more. The term also does not imply that there is no DNA or RNA from other sources. The other source DNA may, for example, comprise DNA from a yeast or bacterial genome, or a cloning vector such as pUCI9. This term distinguishes from naturally occurring events, such as viral infection, or tumor type growths, in which the level of one mRNA may be naturally increased relative to other species of mRNA. That is, the term is meant to cover only those situations in which a person has intervened to elevate the proportion of the desired nucleic acid.

Use of the term “isolated” indicates that a DNA, RNA or protein has been removed from its naturally occurring environment. Thus, the sequence may be in a cell-free solution or placed in a different cellular environment. The term does not imply that the sequence is the only nucleotide chain or polypeptide present, but that it is essentially free (about 90-95% pure at least) of non-nucleotide or non-peptide material naturally associated with it.

It is also advantageous for some purposes that a nucleotide sequence or polypeptide be in purified form, e.g., cloned or recombinant. The term “purified” does not require absolute purity (such as a homogeneous preparation); instead, it represents an indication that the sequence is relatively purer than in the natural environment (compared to the natural level this level should be at least 2-5 fold greater, e.g., in terms of mg/ml).

The nucleic acid molecule may be constructed, for example, from an existing cardiac-function enhancing peptide nucleotide sequence by modification using, for example, oligonucleotide site-directed mutagenesis, by deleting sequences using restriction enzymes, by adding sequences obtained by subcloning or PCR, or as described herein. Standard recombinant techniques for mutagenesis such as in vitro site-directed mutagenesis (Hutchinson et al., J. Biol. Chem. 253:6551, (1978), Sambrook et al., Chapter 15, supra), use of TAB® linkers (Pharmacia), and PCR-directed mutagenesis can be used to create such mutations. The nucleic acid molecule may also be synthesized by the triester method or by using an automated DNA synthesizer.

The invention also features recombinant DNA vectors, preferably in a cell or an organism. The recombinant DNA vectors may contain a sequence coding for a cardiac-function enhancing peptide or a functional derivative thereof in a vector, containing a promoter effective to initiate transcription in a host cell. The recombinant DNA vector may contain a transcriptional initiation region functional in a cell and a transcriptional termination region functional in a cell. Where the DNA vector contains sufficient control sequences, such as initiation and/or termination regions, such that the inserted nucleic acid molecule may be expressed in a host cell, the vector may also be called an “expression vector.”

The present invention also relates to a cell or organism that contains the above-described nucleic acid molecule or recombinant DNA vector and thereby is capable of expressing a cardiac-function enhancing peptide. The peptide may be purified from cells which have been altered to express the polypeptide. A cell is said to be “altered to express a desired polypeptide” when the cell, through genetic manipulation, is made to produce a protein which it normally does not produce or which the cell normally produces at lower levels. One skilled in the art can readily adapt procedures for introducing and expressing either genomic, cDNA, or synthetic sequences into either eukaryotic or prokaryotic cells.

A nucleic acid molecule, such as DNA, is said to be “capable of expressing” a polypeptide if it contains nucleotide sequences which contain transcriptional and translational regulatory information and such sequences are operably linked to nucleotide sequences that encode the polypeptide. The precise nature of the regulatory regions needed for gene sequence expression may vary from organism to organism, but shall in general include a promoter region which, in prokaryotes, contains both the promoter (which directs the initiation of RNA transcription) as well as the DNA sequences which, when transcribed into RNA, will signal synthesis initiation. Such regions will normally include those 5′-non-coding sequences involved with initiation of transcription and translation, such as the TATA box, capping sequence, CAAT sequence, and the like. For example, the entire coding sequence of a cardiac-function enhancing peptide such as, for example, NKX-2.5, may be combined with one or more of the following in an appropriate expression vector to allow for such expression: (1) an exogenous promoter sequence (2) a ribosome binding site (3) a polyadenylation signal (4) a secretion signal (5) a promiscuous or tissue specific enhancer. Combinations of peptides may be generated by combining coding sequences into a transcriptional or translation fusion gene, or the coding sequences may be provided as separate genes or the coding sequences may be provided as separate genes in one or more vectors, preferably in a single vector.

Modifications can be made in the 5′-untranslated and 3′-untranslated sequences to improve expression in a prokaryotic or eukaryotic cell, or codons may be modified such that while they encode an identical amino acid, that codon may be a preferred codon in the chosen expression system. The use of such preferred codons is described in, for example, Grantham et al., Nuc. Acids Res., 9:43-74 (1981), and Lathe, J. Mol. Biol., 183:1-12 (1985) hereby incorporated by reference herein in their entirety. These publications, and all other publications referenced herein, are hereby incorporated by reference in their entirety.

Eukaryotic host cells that may be used in the expression systems of the present invention are not strictly limited, provided that they are suitable for use in the expression of the cardiac-function enhancing peptide of the invention. Preferred eukaryotic hosts include, for example, yeast, fungi, insect cells, mammalian cells either in vivo, or in tissue culture. Mammalian cells which may be useful as hosts include HeLa cells, cells of fibroblast origin such as Vero or CHO-K1, or cells of lymphoid origin and their derivatives.

A wide variety of transcriptional and translational regulatory sequences may be employed, depending upon the nature of the host. The transcriptional and translational regulatory signals may be derived from viral sources, such as adenovirus, bovine papilloma virus, cytomegalovirus, simian virus, or the like, where the regulatory signals are associated with a particular gene sequence which has a high level of expression. Alternatively, promoters from mammalian expression products, such as actin, collagen, myosin, and the like, may be employed. Transcriptional initiation regulatory signals may be selected which allow for repression or activation, so that expression of the gene sequences can be modulated. Of interest are regulatory signals which are temperature-sensitive so that by varying the temperature, expression can be repressed or initiated, or are subject to chemical (such as metabolite) regulation.

Expression of the cardiac-function enhancing peptide of the invention in eukaryotic hosts requires the use of eukaryotic regulatory regions. Such regions will, in general, include a promoter region sufficient to direct the initiation of RNA synthesis. Preferred eukaryotic promoters include, for example, the promoter of the mouse metallothionein I gene sequence (Hamer et al., J. Mol. Appl. Gen. 1:273-288 (1982)); the cytomegalovirus (CMV) promoter, the TK promoter of Herpes virus (McKnight, Cell 31:355-365 (1982)); the SV40 early promoter (Benoist et al., Nature (London) 290:304-310 (1981)); the yeast gal4 gene sequence promoter (Johnston et al., Proc. Natl. Acad. Sci. (USA) 79:6971-6975(1982); Silver et al., Proc. Natl. Acad. Sci. (USA) 81:5951-5955 (1984)).

Translation of eukaryotic mRNA is initiated at the codon which encodes the first methionine. For this reason, it is preferable to ensure that the linkage between a eukaryotic promoter and a DNA sequence that encodes a cardiac-function enhancing peptide or a functional derivative thereof, does not contain any intervening codons which are capable of encoding a methionine (i.e., AUG). The presence of such codons results either in formation of a fusion protein (if the AUG codon is in the same reading frame as, for example, the cardiac-function enhancing peptide protein coding sequence) or a frame-shift mutation (if the AUG codon is not in the same reading frame as, for example, the cardiac-function enhancing peptide protein coding sequence).

A cardiac-function enhancing nucleic acid molecule and an operably linked promoter may be introduced into a recipient prokaryotic or eukaryotic cell either as a nonreplicating DNA (or RNA) molecule, which may either be a linear molecule or, more preferably, a closed covalent circular molecule. Because such molecules are incapable of autonomous replication, the expression of the gene may occur through the transient expression of the introduced sequence. Alternatively, permanent expression may occur through the integration of the introduced DNA sequence into the host chromosome or into a stably-maintained extrachromosomal element. Even if the gene is not incorporated into the chromosome, lack of active cell division tends to maintain expression since there is less loss through cell turnover and “dilution.”

A vector may be employed that is capable of integrating the desired gene sequences into the host cell chromosome. Cells that have stably integrated the introduced DNA into their chromosomes can be selected by also introducing one or more marker genes that allow for selection of host cells which contain the expression vector. The marker may provide for prototrophy in an auxotrophic host, biocide resistance, e.g., antibiotics, or heavy metals, such as copper, or the like. The selectable marker gene sequence can either be directly linked to the DNA gene sequences to be expressed, or introduced into the same cell by co-transfection. Additional elements may also be needed for optimal synthesis of single chain binding protein mRNA. These elements may include splice signals, as well as transcription promoters, enhancers, and termination signals. cDNA expression vectors incorporating such elements include those described by Okayama, Molec. Cell. Biol. 3:280(1983).

The introduced nucleic acid molecule can be incorporated into a plasmid or viral vector capable of autonomous replication in the recipient host. Any of a wide variety of vectors may be employed for this purpose. Factors of importance in selecting a particular plasmid or viral vector include: the ease with which recipient cells that contain the vector may be recognized and selected from those recipient cells which do not contain the vector; the number of copies of the vector which are desired in a particular host and whether it is desirable to be able to “shuttle” the vector between host cells of different species.

Preferred eukaryotic plasmids include, for example, BPV, vaccinia, SV40, 2-micron circle, and the like, or their derivatives. Such plasmids are well known in the art (Botstein et al., Miami Wntr. Symp. 19:265-274(1982); Broach, In: “The Molecular Biology of the Yeast Saccharomyces: Life Cycle and Inheritance”, Cold Spring Harbor Laboratory, Cold Spring Harbor, N.Y., p. 445-470 (1981); Broach, Cell 28:203-204 (1982); Bollon et al., J. Clin. Hematol. Oncol. 10:39-48 (1980); Maniatis, In: Cell Biology: A Comprehensive Treatise, Vol. 3, Gene Sequence Expression, Academic Press, NY, pp. 563-608(1980).

Presently preferred replication-defective adenoviral vectors have been changed to inactivate or delete the E1A and E1B genes, the E1A, E1B and E4 genes, the E1A, E1B and E3 genes, or the E1A, E1B and E2 genes. Preferably about 10¹⁰ to 10¹⁴ adenovirus vector particles, more preferably about 10¹¹ to 10¹³ vector particles, most preferably about 10¹² vector particles, are introduced into a blood vessel, preferably a blood vessel supplying the myocardium.

For AAV vectors, the vector preferably comprises a polynucleotide having a promoter operably linked to a gene encoding a cardiac function-enhancing protein or peptide, and preferably, the gene encoding the protein or peptide is flanked by AAV inverted terminal repeats (ITRs). Preferably, the AAV vector is replication-defective in humans. Presently preferred replication-defective AAV vectors have deletions affecting one or more AAV replication or encapsidation sequences, more preferably all of the replication and encapsidation sequences are removed.

Alternatively, the vector can be a lipid-based vector comprising a gene encoding a cardiac function-enhancing protein or peptide as described herein. Numerous lipid-based vectors have been described in the art and many are commercially available.

A “promoter,” as used herein, refers to a polynucleotide sequence that controls transcription of a gene or coding sequence to which it is operably linked. A large number of promoters, including constitutive, inducible and repressible promoters, from a variety of different sources, are well known in the art and are available as or within cloned polynucleotide sequences (from, e.g., depositories such as the ATCC as well as other commercial or individual sources).

An “enhancer,” as used herein, refers to a polynucleotide sequence that enhances transcription of a gene or coding sequence to which it is operably linked. A large number of enhancers, from a variety of different sources are well known in the art and available as or within cloned polynucleotide sequences (from, e.g., depositories such as the ATCC as well as other commercial or individual sources). A number of polynucleotides comprising promoter sequences (such as the commonly-used CMV promoter) also comprise enhancer sequences.

“Operably linked” refers to a juxtaposition, wherein the components so described are in a relationship permitting them to function in their intended manner. A promoter is operably linked to a coding sequence if the promoter controls transcription of the coding sequence. Although an operably linked promoter is generally located upstream of the coding sequence, it is not necessarily contiguous with it. An enhancer is operably linked to a coding sequence if the enhancer increases transcription of the coding sequence. Operably linked enhancers can be located upstream, within, or downstream of coding sequences. A polyadenylation sequence is operably linked to a coding sequence if it is located at the downstream end of the coding sequence such that transcription proceeds through the coding sequence into the polyadenylation sequence. A gene, or a transgene, is driven by a promoter where the promoter controls transcription of the gene or transgene.

An operable linkage is thus a linkage in which the regulatory DNA sequences and the DNA sequence sought to be expressed are connected in such a way as to permit gene sequence expression. Two DNA sequences (such as a promoter region sequence and a cardiac-function enhancing peptide sequence) are said to be operably linked if the nature of the linkage between the two DNA sequences does not (1) result in the introduction of a frame-shift mutation in the coding sequence, (2) interfere with the ability of the promoter region sequence to direct the transcription of cardiac-function enhancing peptide gene sequence, or (3) interfere with the ability of the cardiac-function enhancing peptide gene sequence to be transcribed by the promoter region sequence. Thus, a, promoter region would be operably linked to a DNA sequence if the promoter were capable of effecting transcription of that DNA sequence. Thus, to express a cardiac-function enhancing peptide transcriptional and translational signals recognized by an appropriate host are necessary.

“Gene delivery,” “gene transfer,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

“In vivo” gene delivery, gene transfer, gene therapy and the like as used herein, are terms referring to the introduction of a vector comprising an exogenous polynucleotide directly into the body of an organism, such as a human or non-human mammal, whereby the exogenous polynucleotide is introduced to a cell of such organism in vivo.

A “recombinant viral vector” refers to a viral vector comprising one or more heterologous genes or sequences. Since many viral vectors exhibit size-constraints associated with packaging, the heterologous genes or sequences are typically introduced by replacing one or more portions of the viral genome. Such viruses may become replication-defective, requiring the deleted function(s) to be provided in trans during viral replication and encapsulation (by using, e.g., a helper virus or a packaging cell line carrying genes necessary for replication and/or encapsulation) (see, e.g., the references and illustrations below). Modified viral vectors in which a polynucleotide to be delivered is carried on the outside of the viral particle have also been described (see, e.g., Curiel, D T, et al. PNAS 88: 8850-8854, 1991).

Viral “packaging” as used herein refers to a series of intracellular events that results in the synthesis and assembly of a viral vector. Packaging typically involves the replication of the “pro-viral genome”, or a recombinant pro-vector typically referred to as a “vector plasmid” (which is a recombinant polynucleotide than can be packaged in an manner analogous to a viral genome, typically as a result of being flanked by appropriate viral “packaging sequences”), followed by encapsulation or other coating of the nucleic acid. Thus, when a suitable vector plasmid is introduced into a packaging cell line under appropriate conditions, it can be replicated and assembled into a viral particle. Viral “rep” and “cap” genes, found in many viral genomes, are genes encoding replication and encapsulation proteins, respectively. A “replication-defective” or “replication-incompetent” viral vector refers to a viral vector in which one or more functions necessary for replication and/or packaging are missing or altered, rendering the viral vector incapable of initiating viral replication following uptake by a host cell. To produce stocks of such replication-defective viral vectors, the virus or pro-viral nucleic acid can be introduced into a “packaging cell line” that has been modified to contain genes encoding the missing functions which can be supplied in trans. For example, such packaging genes can be stably integrated into a replicon of the packaging cell line or they can be introduced by transfection with a “packaging plasmid” or helper virus carrying genes encoding the missing functions.

A “detectable marker gene” is a gene that allows cells carrying the gene to be specifically detected (e.g., distinguished from cells which do not carry the marker gene). A large variety of such marker genes are known in the art. Preferred examples thereof include detectable marker genes which encode proteins appearing on cellular surfaces, thereby facilitating simplified and rapid detection and/or cellular sorting. By way of illustration, the lacZ gene encoding beta-galactosidase can be used as a detectable marker, allowing cells transduced with a vector carrying the lacZ gene to be detected by staining, as described below.

A “selectable marker gene” is a gene that allows cells carrying the gene to be specifically selected for or against, in the presence of a corresponding selective agent. By way of illustration, an antibiotic resistance gene can be used as a positive selectable marker gene that allows a host cell to be positively selected for in the presence of the corresponding antibiotic. Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e., positive/negative) markers (see, e.g., WO 92/08796, published May 29, 1992, and WO 94/28143, published Dec. 8, 1994), hereby incorporated by reference herein. Such marker genes can provide an added measure of control that can be advantageous in gene therapy contexts.

“Vasculature” or “vascular” are terms referring to the system of vessels carrying blood (as well as lymph fluids) throughout the mammalian body.

“Blood vessel” refers to any of the vessels of the mammalian vascular system, including arteries, arterioles, capillaries, venules, veins, sinuses, and vasa vasorum.

“Artery” refers to a blood vessel through which blood passes away from the heart. Coronary arteries supply the tissues of the heart itself, while other arteries supply the remaining organs of the body. The general structure of an artery consists of a lumen surrounded by a multi-layered arterial wall.

An “individual” or “patient” refers to a mammal, preferably a large mammal, most preferably a human.

“Treatment” or “therapy” as used herein refers to administering, to an individual patient, agents that are capable of eliciting a prophylactic, curative or other beneficial effect on the individual.

“Gene therapy” as used herein refers to administering, to an individual patient, vectors comprising a therapeutic gene.

A “therapeutic polynucleotide” or “therapeutic gene” refers to a nucleotide sequence that is capable, when transferred to an individual, of eliciting a prophylactic, curative or other beneficial effect in the individual.

References

The practice of the present invention will employ, unless otherwise indicated, conventional techniques of molecular biology and the like, which are within the skill of the art. Such techniques are explained fully in the literature. See e.g., Molecular Cloning: A Laboratory Manual, (J. Sambrook et al., Cold Spring Harbor Laboratory, Cold Spring Harbor, N.Y., 1989); Current Protocols in Molecular Biology (F. Ausubel et al. eds., 1987 and updated); Essential Molecular Biology (T. Brown ed., IRL Press 1991); Gene Expression Technology (Goeddel ed., Academic Press 1991); Methods for Cloning and Analysis of Eukaryotic Genes (A. Bothwell et al. eds., Bartlett Publ. 1990); Gene Transfer and Expression (M. Kriegler, Stockton Press 1990); Recombinant DNA Methodology (R. Wu et al. eds., Academic Press 1989); PCR: A Practical Approach (M. McPherson et al., IRL Press at Oxford University Press 1991); Cell Culture for Biochemists (R. Adams ed., Elsevier Science Publishers 1990); Gene Transfer Vectors for Mammalian Cells (J. Miller & M. Calos eds., 1987); Mammalian Cell Biotechnology (M. Butler ed., 1991); Animal Cell Culture (J. Pollard et al. eds., Humana Press 1990); Culture of Animal Cells, 2nd Ed. (R. Freshney et al. eds., Alan R. Liss 1987); Flow Cytometry and Sorting (M. Melamed et al. eds., Wiley-Liss 1990); the series Methods in Enzymology (Academic Press, Inc.); Techniques in Immunocytochemistry, (G. Bullock & P. Petrusz eds., Academic Press 1982, 1983, 1985, 1989); Handbook of Experimental Immunology, (D. Weir & C. Blackwell, eds.); Cellular and Molecular Immunology (A. Abbas et al., W. B. Saunders Co. 1991, 1994); Current Protocols in Immunology (J. Coligan et al. eds. 1991); the series Annual Review of Immunology; the series Advances in Immunology; Oligonucleotide Synthesis (M. Gait ed., 1984); and Animal Cell Culture (R. Freshney ed., IRL Press 1987).

Additional references describing delivery and logistics of surgery which may be used in the methods of the present invention include the following: Topol, E J (ed.), The Textbook of Interventional Cardiology, 2nd Ed. (W. B. Saunders Co. 1994); Rutherford, R B, Vascular Surgery, 3rd Ed. (W. B. Saunders Co. 1989); The Cecil Textbook of Medicine, 19th Ed. (W.B. 1992); and Sabiston, D, The Textbook of Surgery, 14th Ed. (W.B. 1991). Additional references describing cell types found in the blood vessels, and those of the vasculature which may be useful in the methods of the present invention include the following: W. Bloom & D. Fawcett, A Textbook of Histology (V. B. Saunders Co. 1975).

Various publications have postulated on the uses of gene transfer for the prevention of disease, including heart disease. See, e.g., Methods in Virology, Vol. 7: Gene Transfer and Expression Protocols, Murray, E. (ed.), Weiss, Clifton, N.J., 1991; Mazur et al., Molecular and Cellular Biology, 21:104-111, 1994; French, Herz 18:222-229, 1993; Williams, Journal of Medical Sciences 306:129-136, 1993; and Schneider, Circulation 88:1937-1942, 1993.

All of the references cited in this application are incorporated by reference herein to the extent that these references describe compositions and techniques that can be employed in the practice of the present invention.

Description of Various Preferred Embodiments

Various preferred aspects of the present invention are summarized below and described and illustrated in the subsequent detailed descriptions.

Animal Models:

Animal models may be used to evaluate the effects of gene transfer. One model, as in Example 5, mimics clinical congestive heart failure. In another model, for purposes of evaluating congestive heart failure and myocardial infarction, dogs may be used instead of pigs. Coronary occlusion models resulting in myocardial infarction can be used to evaluate the effects of gene transfer on heart muscle regeneration, remodeling, and congestive heart failure.

Those skilled in the art will recognize that these and other animal models can provide useful data on the effects of cardiac function-enhancing peptides in the heart.

Models of Myocardial Infarction:

Models of acute myocardial infarction or myocardial necrosis have been developed in several species, primarily dog, pig, rabbit and rat. Those skilled in the art will be readily familiar with the use of these models. In one model, myocardial infarction occurs under general anesthesia. The animal is anesthetized, intubated, and ventilated. The chest is opened through a sternotomy or a lateral thoracotomy. The appropriate branch of coronary artery is next identified. This can be the left anterior descending coronary artery, the circumflex coronary artery, or the anterior artery. The coronary artery is next ligated with a suture. With some animal models, those practiced in the art will know to ligate the artery in stages. In some species, such as the dog, pre-existing collateral vessels may also have to be ligated. The vessel may be left permanently ligated, or reperfusion accomplished in some embodiments, by opening the vessel after a sustained period of occlusion. Reopening the vessel provides a model of myocardial infarction with reperfusion.

In another application of the model, coronary occlusion occurs in conscious animals. Here, the animals are anesthetized first and instruments implanted at thoracotomy. A coronary artery balloon occluder is placed around the vessel to be occluded. Appropriate wires, instruments, and the catheter to the balloon occluder are tunneled subcutaneously to a place usually behind the neck. When the animal has recovered sufficiently, usually a few days, the coronary artery can be occluded, or occluded and reperfused, in a conscious state.

In a third type of model, the myocardial infarction or necrosis is generated by a means other than ischemia. In one model, a metal paddle cooled in liquid nitrogen is placed on the epicardial surface. See, e.g. Murry, C. E. et al., J Clin Invest (1996) 98:2209-2217.

In another form of the model, intense electrical stimulation by a large voltage results in myocardial necrosis. Following formation of myocardial infarction, gene transfer is accomplished by methods described herein. Histologic analysis can be used to determine phenotype switch to, e.g., cardiac myocytes.

Several models of congestive heart failure have been developed. In one preferred model, rapid pacing of the ventricle for sustained periods up to four weeks is performed. At thoracotomy under general anesthesia, a cardiac pacemaker lead is placed on the ventricle of the animal and tunneled to a subcutaneous pocket where a pacemaker is implanted. Following recovery, the pacemaker can be activated and over a period of one to four weeks, rapid pacing results in congestive heart failure. The features of congestive heart failure in this model mimic those seen in patients with chronic congestive heart failure (See, e.g., Hammond et al. WO98/10085, published Mar. 12, 1998, and WO98/50079, published Nov. 12, 1998.)

In yet another model of chronic congestive heart failure, multiple coronary emboli are delivered to the coronary arteries to produce myocardial infarction. Under light general anesthesia at cardiac catheterization, beads the size of 25-100 μm diameter, or in some embodiments up to 250 μm diameter, are embolized down the coronary artery until a sustained elevation in diastolic pressure of the left ventricle is achieved. The catheter is then withdrawn and the animals are allowed to recover. Animals develop congestive heart failure over the ensuing weeks with all of the clinical manifestations of chronic congestive heart failure in patients.

Once stable congestive heart failure is established, gene therapy as described herein, for example with growth hormone or anti-apoptotic protein genes, is performed. An analysis will reveal an improvement in the state of congestive heart failure compared to placebo-treated controls.

Gene Therapy:

Nucleic acids coding for a cardiac-function enhancing peptide will also be useful in gene therapy. In vivo gene therapy applications and methodologies are illustrated in the patents and patent applications by Hammond et al., supra, and are further illustrated herein.

As another example, an expression vector containing the cardiac-function enhancing peptide coding sequence may be inserted into cells, the cells are grown in vitro and then infused in large numbers into patients. In another example, a DNA segment containing a promoter of choice (for example a strong promoter) is transferred into cells containing an endogenous cardiac-function enhancing peptide should such endogenous forms exist, in such a manner that the promoter segment enhances expression of the endogenous cardiac-function enhancing peptide gene (for example, the promoter segment is transferred to the cell such that it becomes directly linked to the endogenous cardiac-function enhancing peptide gene), or, a protein (or gene encoding it) which turns on the endogenous promoter can be provided as an alternative means of stimulating expression of an endogenous gene.

The gene therapy may involve the use of an adenovirus vector including a nucleotide sequence coding for a cardiac-function enhancing peptide or a naked nucleic acid molecule coding for these proteins. Alternatively, engineered cells containing a nucleic acid molecule coding for these modified proteins may be injected.

Expression vectors derived from viruses such as retroviruses, vaccinia virus, adenovirus, adeno-associated virus, herpes viruses, several RNA viruses, or bovine papilloma virus, may be used for delivery of nucleotide sequences (e.g., cDNA) encoding a recombinant cardiac-function enhancing peptide into the targeted cell population. Methods which are well known to those skilled in the art can be used to construct recombinant viral vectors containing coding sequences. See, for example, Nabel, E. G., Circulation, 91, 541-548 (1995), the techniques described in Maniatis et al., Molecular Cloning: A Laboratory Manual, Cold Spring Harbor Laboratory, N.Y. (1989), and in Ausubel et al., Current Protocols in Molecular Biology, Greene Publishing Associates and Wiley Interscience, N.Y. (1989). Alternatively, recombinant nucleic acid molecules encoding protein sequences can be used as naked DNA or in reconstituted system e.g., liposomes or other lipid systems for delivery to target cells (See e.g., Felgner et al., Nature 337:397-8, 1989). Several other methods for the direct transfer of plasmid DNA into cells exist for use in human gene therapy and involve targeting the DNA to receptors on cells by complexing the plasmid DNA to proteins. See, Miller, Nature 357:455-60, 1992.

In its simplest form, gene transfer can be performed by simply injecting minute amounts of DNA into the nucleus of a cell, through a process of microinjection. Capecchi, M. R., Cell 22:479-88 (1980). Once recombinant genes are introduced into a cell, they can be recognized by the cell's normal mechanisms for transcription and translation, and a gene product will be expressed. Other methods have also been attempted for introducing DNA into larger numbers of cells. These methods include: transfection, wherein DNA is precipitated with CaPO₄ and taken into cells by pinocytosis (Chen, C. and Okayama, H., Mol. Cell Biol. 7:2745-52 (1987)); electroporation, wherein cells are exposed to large voltage pulses to introduce holes into the membrane (Chu, G. et al., Nucleic Acids Res., 15:1311-26 (1987)); lipofection/liposome fusion, wherein DNA is packaged into lipophilic vesicles which fuse with a target cell (Felgner, P. L., et al., Proc. Natl. Acad. Sci. USA. 84:7413-7 (1987)); and particle bombardment using DNA bound to small projectiles (Yang, N. S., et al., Proc. Natl. Acid Sci. 87:9568-72 (1990)). Another method for introducing DNA into cells is to couple the DNA to chemically modified proteins.

It has also been shown that adenovirus proteins are capable of destabilizing endosomes and enhancing the uptake of DNA into cells. The admixture of adenovirus to solutions containing DNA complexes, or the binding of DNA to polylysine covalently attached to adenovirus using protein crosslinking agents substantially improves the uptake and expression of the recombinant gene. Curiel, D. T., et al., Am. J. Respir. Cell. Mol. Biol. 6:247-52 (1992).

As used herein “gene transfer” means the process of introducing a foreign nucleic acid molecule into a cell. Gene transfer is commonly performed to enable the expression of a particular product encoded by the gene. The product may include a protein, polypeptide, anti-sense DNA or RNA, or enzymatically active RNA. Gene transfer can be performed in cultured cells or by direct administration into animals. Generally gene transfer involves the process of nucleic acid molecule contact with a target cell by non-specific or receptor mediated interactions, uptake of nucleic acid molecule into the cell through the membrane or by endocytosis, and release of nucleic acid molecule into the cytoplasm from the plasma membrane or endosome. Expression may require, in addition, movement of the nucleic acid molecule into the nucleus of the cell and binding to appropriate nuclear factors for transcription.

As used herein “gene therapy” is a form of gene transfer and is included within the definition of gene transfer as used herein and specifically refers to gene transfer to express a therapeutic product from a cell in vivo or in vitro. Gene transfer can be performed ex vivo on cells which are then transplanted into a patient, or can be performed by direct administration of the nucleic acid molecule or nucleic acid-protein complex into the patient.

In another preferred embodiment, a vector having nucleic acid molecule sequences encoding a cardiac-function enhancing peptide is provided in which the nucleic acid molecule sequence is expressed only in a specific tissue. Methods of achieving tissue-specific gene expression are set forth in International Publication No. WO 93/09236, filed Nov. 3, 1992 and published May 13, 1993.

In all of the preceding vectors set forth above, a further aspect of the invention is that the nucleic acid sequence contained in the vector may include additions, deletions or modifications to some or all of the sequence of the nucleic acid, as defined above.

In another preferred embodiment, a method of gene replacement is set forth. “Gene replacement” as used herein means supplying a nucleic acid molecule sequence which is capable of being expressed in vivo in an animal and thereby providing or augmenting the function of an endogenous gene which is missing or defective in the animal.

Vectors for Gene Delivery In Vivo:

In general, the gene of interest is transferred to the target organ, preferably the heart, including cardiac myocytes, myofibroblasts, endothelial cells, and/or fibroblasts and directs production of the encoded protein. Preferably such production is relatively constitutive. A variety of different gene transfer vectors, including viral as well as non-viral systems, can be employed to deliver transgenes for use in the present invention.

Preferred vectors for use in the present invention include viral vectors, lipid-based vectors and other vectors that are capable of delivering DNA to non-dividing cells in vivo. Presently preferred are viral vectors, particularly replication-defective viral vectors (including, for example replication-defective adenovirus vectors and adeno-associated virus (AAV) vectors. For ease of production and use in the present invention, replication-defective adenovirus vectors are presently most preferred.

References describing a variety of other gene delivery vectors are known in the art, some of which are cited herein. Such other vectors include, for example, other viral vectors (such as adeno-associated viruses (AAV), liposomes and other lipid-containing complexes, and other macromolecular complexes capable of mediating delivery of a polynucleotide to a host cell. As described above and in the cited references, vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Selectable markers can be positive, negative or bifunctional. Positive selectable markers allow selection for cells carrying the marker, whereas negative selectable markers allow cells carrying the marker to be selectively eliminated.

Additional references describing adenovirus vectors and other viral vectors which could be used in the methods of the present invention include the following: Horwitz, M. S., Adenoviridae and Their Replication, in Fields, B., et al. (eds.) Virology, Vol. 2, Raven Press New York, pp. 1679-1721, 1990); Graham, F., et al., pp. 109-128 in Methods in Molecular Biology, Vol. 7: Gene Transfer and Expression Protocols, Murray, E. (ed.), Humana Press, Clifton, N.J. (1991); Miller, N., et al., FASEB Journal 9: 190-199, 1995; Schreier, H, Pharmaceutica Acta Heivetiae 68: 145-159, 1994; Schneider and French, Circulation 88:1937-1942, 1993; Curiel D. T., et al., Human Gene Therapy 3: 147-154, 1992; Graham, F. L., et al., WO 95/00655 (Jan. 5, 1995); Falck-Pedersen, E. S., WO 95/16772 (Jun. 22, 1995); Denefle, P. et al., WO 95/23867 (Sep. 8, 1995); Haddada, H. et al., WO 94/26914 (Nov. 24, 1994); Perricaudet, M. et al., WO 95/02697 (Jan. 26, 1995); Zhang, W., et al., WO 95/25071 (Oct. 12, 1995). A variety of adenovirus plasmids are also available from commercial sources, including, e.g., Microbix Biosystems of Toronto, Ontario (see, e.g., Microbix Product Information Sheet: Plasmids for Adenovirus Vector Construction, 1996).

Additional references describing AAV vectors which could be used in the methods of the present invention include the following: Carter, B., Handbook of Parvoviruses, vol. 1, pp. 169-228, 1990; Berns, Virology, pp. 1743-1764 (Raven Press 1990); Carter, B., Curr. Opin. Biotechnol., 3: 533-539, 1992; Muzyczka, N., Current Topics in Microbiology and Immunology, 158: 92-129, 1992; Flotte, T. R., et al., Am. J. Respir. Cell Mol. Biol. 7:349-356, 1992; Chatterjee et al., Ann. NY Acad. Sci., 770: 79-90, 1995; Flotte, T. R., et al., WO 95/13365 (May 18, 1995); Trempe, J. P., et al., WO 95/13392 (May 18, 1995); Kotin, R., Human Gene Therapy, 5: 793-801, 1994; Flotte, T. R., et al., Gene Therapy 2:357-362, 1995; Allen, J. M., WO 96/17947 (Jun. 13, 1996); and Du et al., Gene Therapy 3: 254-261, 1996.

Lentiviruses, which can also be employed for delivery of genes in the context of the present invention, have been described, see, e.g., Blomer et al., J. Virol., 71:6641-6649 (1997), Kafri et al., Nature Genet. 17:314-317 (1997); and Poeschla et al., J. Virol. 72: 6527-36 (1998).

Additional references describing non-viral vectors which could be used in the methods of the present invention include the following: Ledley, F D, Human Gene Therapy 6:1129-1144, 1995; Miller, N., et al., FASEB Journal 9:190-199, 1995; Chonn, A., et al., Curr. Opin. in Biotech. 6:698-708, 1995; Schofield, J P, et al., British Med. Bull. 51: 56-71, 1995; Brigham, K. L., et al., J. Liposome Res. 3:31-49, 1993; Brigham, K. L., WO 91/06309 (May 16, 1991); Felgner, P. L., et al., WO 91/17424 (Nov. 14, 1991); Solodin et al., Biochemistry 34: 13537-13544, 1995; WO 93/19768 (Oct. 14, 1993); Debs et al., WO 93125673; Felgner, P. L., et al., U.S. Pat. No. 5,264,618 (Nov. 23, 1993); Epand, R. M., et al., U.S. Pat. No. 5,283,185 (Feb. 1, 1994); Gebeyehu et al., U.S. Pat. No. 5,334,761 (Aug. 2, 1994); Felgner, P. L., et al., U.S. Pat. No. 5,459,127 (Oct. 17, 1995); Overell, R. W., et al., WO 95/28494 (Oct. 26, 1995); Jessee, WO 95/02698 (Jan. 26, 1995); Haces and Ciccarone, WO 95/17373 (Jun. 29, 1995); Lin et al., WO 96/01840 (Jan. 25, 1996).

Helper Independent Replication Deficient Human Adenovirus 5 System:

In general, the gene of interest is transferred to the heart, including cardiac myocytes, fibroblasts, myofibroblasts, endothelial cells, or other cells within the heart, in vivo and directs constitutive production of the encoded protein. Several different gene transfer approaches are feasible. Presently preferred is the helper-independent replication deficient human adenovirus 5 system. Using this system, transfection of greater than 60% of myocardial cells has been demonstrated in vivo by a single intracoronary injection (Giordano, F. J. et al., Nature Med 2:534-39, 1996). Non-replicative recombinant adenoviral vectors are particularly useful in transfecting coronary endothelium and cardiac myocytes resulting in highly efficient transfection after intracoronary injection.

The helper-independent replication-defective human adenovirus 5 system can be used effectively to transfect a large percentage of myocardial cells in vivo by a single intracoronary injection. Such a delivery technique may be used to effectively target vectors to the myocardium of a large mammal heart. Additional means of targeting vectors to particular cells or tissue types are described below and in the art.

In various illustrations described below, recombinant adenovirus vectors based on the human adenovirus 5 are used (as described by McGrory W J, et al., Virology 163:614-617, 1988) which are defective in essential early genes from the adenovirus genome (usually E1A, E1B, E2, E3 and/or E4), and are therefore unable to replicate unless grown in permissive cell lines that provide the missing gene products in trans. In place of the missing adenovirus genomic sequences, a transgene of interest can be cloned and expressed in tissue/cells infected with the replication-defective adenovirus. Although adenovirus-based gene transfer does not generally result in stable integration of the transgene into the host genome, adenovirus vectors can be propagated in high titer and transfect non-replicating cells; and, although the transgene does not replicate during the S-phase of the cell cycle it is suitable for gene transfer to adult cardiac myocytes, which do not actively divide. Retrovirus vectors provide stable gene transfer, and high titers are now obtainable via retrovirus pseudotyping (Burns, et al., Proc. Natl. Acad. Sci. (USA) 90: 8033-8037, 1993), but current retrovirus vectors are generally unable to efficiently transduce nonreplicating cells.

An advantage associated with nondividing cells such as myocytes is that the viral vector is not readily “diluted out” by host cell division. To further enhance the duration of transgene expression in the heart, however, it is also possible to employ various second generation adenovirus vectors that have both E1 and E4 deletions, which can be used in conjunction with cyclophosphamide administration (See, e.g., Dai et al., Proc. Natl. Acad. Sci. (USA) 92: 1401-1405, 1995). To further increase the extent of initial gene transfer, multiple infusions, or infusion in an isolated coronary circuit can also be employed.

Transfection of replicating cells in the heart, such as fibroblasts, is also achieved by adenoviral gene transfer. When such cells are subjected to gene transfer with a gene capable of inducing a phenotype switch, subsequent daughter cells can express the desired phenotype.

Human 293 cells, which are human embryonic kidney cells transformed with adenovirus E1A and E1B genes, typify useful permissive cell lines for the production of such replication-defective vectors. However, other cell lines which allow replication-defective adenovirus vectors to propagate therein can also be used, such as HeLa cells, HEK293 cells, and PERC6 cells (IntroGene).

By way of illustration, the recombinant adenoviral vectors based on the human adenovirus 5 (Virology, 163:614-617, 1988) are missing essential early genes from the adenoviral genome (usually E1A/E1B), and are therefore unable to replicate unless grown in permissive cell lines that provide the missing gene products in trans. In place of the missing adenoviral genomic sequences, a transgene of interest can be cloned and expressed in tissue/cells infected with the replication deficient adenovirus. Although adenovirus-based gene transfer does not result in integration of the transgene into the host genome (less than 0.1% adenovirus-mediated transfections result in transgene incorporation into host DNA), and therefore is not stable, adenoviral vectors can be propagated in high titer and transfect non-replicating cells. Although the transgene is not passed to daughter cells, this is acceptable for gene transfer to adult skeletal muscle and cardiac myocytes, which do not divide. Retroviral vectors provide stable gene transfer, and high titers are now obtainable via retrovirus pseudotyping (Burns, et al., Proc. Natl. Acad. Sci. (USA), 90:8033-8037, 1993), but current retroviral vectors are unable to transduce nonreplicating cells (adult skeletal muscle and cardiac myocytes) efficiently.

Human 293 cells, which are human embryonic kidney cells transformed with adenovirus E1A/E1B genes, typify useful permissive cell lines. However, other cell lines which allow replication-deficient adenoviral vectors to propagate therein can be used.

Construction of Recombinant Adenoviral Vectors:

All adenoviral vectors used in the present invention may be constructed by the rescue recombination technique described in Graham, Virology, 163:614-617, 1988. Briefly, the transgene of interest is cloned into a shuttle vector that contains a promoter, polylinker and partial flanking adenoviral sequences from which E1A and E1B genes have been deleted. As the shuttle vector, plasmid pAC1 (Virology, 163:614-617, 1988) (or an analog) which encodes portions of the left end of the human adenovirus 5 genome (Virology, 163:614-617, 1988) minus the early protein encoding E1A and E1B sequences that are essential for viral replication, and plasmid ACCMVPLPA (J. Biol. Chem., 267:25129-25134, 1992) which contains polylinker, the CMV promoter and SV40 polyadenylation signal flanked by partial adenoviral sequences from which the E1A and E1B genes have been deleted can be exemplified. The use of plasmid pAC1 or ACCMVPLA facilitates the cloning process. The shuttle vector is then co-transfected with a plasmid which contains the entire human adenoviral 5 genome with a length too large to be encapsidated, into 293 cells. Co-transfection can be conducted by calcium phosphate precipitation or lipofection (Biotechniques, 15:868-872, 1993). Plasmid JM17 encodes the entire human adenovirus 5 genome plus portions of the vector pBR322 including the gene for ampicillin resistance (4.3 kb). Although JM17 encodes all of the adenoviral proteins necessary to make mature viral particles, it is too large to be encapsidated (40 kb versus 36 kb for wild type). In a small subset of co-transfected cells, rescue recombination between the transgene containing the shuttle vector such as plasmid pAC1 and the plasmid having the entire adenoviral 5 genome such as plasmid pJM17 provides a recombinant genome that is deficient in the E1A and E1B sequences, and that contains the transgene of interest but secondarily loses the additional sequence such as the pBR322 sequences during recombination, thereby being small enough to be encapsidated. With respect to the above method, successful results have been reported (Giordano, F. J. et al., Nature Med 2:534-39, 1996; Giordano, et al., Circulation, 88:I-139, 1993, and Giordano and Hammond, Clin. Res., 42:123A, 1994). The CMV driven β-galactosidase encoding adenovirus HCMVSP11acZ (Clin. Res., 42:123A, 1994) can be used to evaluate efficiency of gene transfer using X-gal treatment.

The initial mode of gene transfer uses adenoviral vectors as delineated above. The advantages of these vectors include the ability to effect high efficiency gene transfer (more than 60% of target organ cells transfected in vivo), the ease of obtaining high titer viral stocks and the ability of these vectors to effect gene transfer into cells such as cardiac myocytes which do not divide.

Tissue-Specific Promoters:

The present invention also contemplates the use of cell targeting not only by delivery of the transgene into the coronary artery, but also the use of tissue-specific promoters, such as, for example, heart cell-specific promoters. By fusing, for example, tissue-specific transcriptional control sequences of left ventricular myosin light chain-2 (MLC₂ᵥ), or myosin heavy chain (MHC) or another cardiac-specific transcriptional control sequence to a transgene such as a cardiac-function enhancing peptide gene within the adenoviral construct, transgene expression is limited to ventricular cardiac myocytes. The efficacy of gene expression and degree of specificity provided by MDC₂ᵥ and MHC promoters with lacZ have been determined, using the recombinant adenoviral system of the present invention. Cardiac-specific expression has been reported previously by Lee, et al. (J. Biol. Chem., 267:15875-15885, 1992). The MLC₂ᵥ promoter is comprised of 250 bp, and fits easily within the adenoviral-5 packaging constraints. The myosin heavy chain promoter, known to be a vigorous promoter of transcription, provides a reasonable alternative cardiac-specific promoter and is comprised of less than 300 bp. Other promoters, such as the troponin-C promoter, while highly efficacious and sufficiently small, lack adequate tissue specificity. By using the MLC₂ᵥ or MHC promoters and delivering the transgene in vivo, it is believed that the cardiac myocyte alone (that is without concomitant expression in endothelial cells, smooth muscle cells, and fibroblasts within the heart) will provide adequate expression of a cardiac function enhancing peptide to enhance cardiac function. Limiting expression to the cardiac myocyte also has advantages regarding the utility of gene transfer for the treatment of CHF. By limiting expression to the heart, one avoids the potentially harmful effect of protein production in non-cardiac tissues such as the retina and provides some element of organ targeting. If the cardiac function-enhancing protein is a hormone, concentration will be highest in the heart and significantly lower by dilution in blood and other organs, thus limiting unwanted side effects or toxic effects. In addition, of the cells in the heart, the myocyte would likely provide the longest transgene expression since the cells do not undergo rapid turnover. Endothelial-specific promoters and smooth muscle cell promoters are already available for this purpose (Lee, et al., J Biol Chem, 265:10446-10450, 1990; Kim, S., J Clin Invest 100:1006-14 (1997)). Other specific promoters may be used to target fibroblasts or myofibroblasts that may be located in the heart.

In the present invention, with regard to the treatment of heart disease, targeting the heart by intracoronary injection with a high titer of the vector and transfecting all cell types is presently preferred.

Propagation and Purification of Adenovirus Vectors:

Successful recombinant vectors can be plaque purified according to standard methods. The resulting viral vectors are propagated on 293 cells which provide E1A and E1B functions in trans, to titers in the preferred 10¹⁰-10¹² viral particles/ml range. Cells can be infected at 80% confluence and harvested 48 hours later. After 3 freeze-thaw cycles the cellular debris is pelleted by centrifugation and the virus purified by CsCl gradient ultracentrifugation (double CsCl gradient ultracentrifugation (or column chromatography) is preferred. Prior to in vivo injection, the viral stocks are desalted by gel filtration through Sepharose columns such as G25 Sephadex. The product may also be filtered (e.g., through a 0.3 micron filter). The resulting viral stock typically has a final viral titer in the range of 10¹⁰-10¹² viral particles/ml. The recombinant adenovirus should be highly purified, with few wild-type (potentially replicative) virus. Preferably fewer than 1 replication competent particle per million, more preferably fewer than 1 per 10⁹, most preferably fewer than 1 per 10¹². From this point of view, propagation and purification may be conducted to exclude contaminants and wild-type virus by, for example, identifying successful recombinants with PCR using appropriate primers, conducting two rounds of plaque purification, and double CsCl gradient ultracentrifugation.

Delivery of Recombinant Adenovirus Vectors:

The viral stock can be in the form of an injectable preparation containing pharmaceutically acceptable carrier such as saline, for example, as necessary. The final titer of the vector in the injectable preparation is preferably in the range of 10⁷-10¹³ viral particles which allows for effective gene transfer. Other pharmaceutical carriers, formulations and dosages are described below. The adenovirus transgene constructs can be delivered to the myocardium by direct intracoronary (or graft vessel) injection or coronary sinus retroprofusion using standard percutaneous catheter based methods under fluoroscopic guidance, at an amount sufficient for the transgene to be expressed to a degree which allows for highly effective therapy. The injection should be made deeply into the lumen (about 1-3 cm within the arterial lumen) of the coronary arteries (or graft vessel), and preferably be made in one or more coronary arteries (see, e.g., Hammond et al., U.S. Pat. No. 5,792,453), or by coronary sinus retroprofusion as described in the art (see, e.g., Gore, J., et al., Circ. 74-381(1986); Meerbaum, S., et al., J. Am. Coll. Cardiol. 1:1262-7 (1983); Povzhitkov, M., J. Am. Coll. Cardiol. 3:939-47 (1984)). Histamine or another vasoactive agent can also be used to enhance intravascular gene delivery, as described by Hammond et al. in international application PCT/US99/02702, filed Feb. 9, 1999.

By injecting the material directly into the lumen of the coronary artery by coronary catheters, it is possible to target the gene rather effectively, and to minimize loss of the recombinant vectors to the proximal aorta during injection. The vector stock containing virus may be injected deeply into the lumen of one or more coronary arteries (or grafts), preferably into both right and left coronary arteries (or grafts), and preferably in an amount of 10¹⁰-10¹⁴ viral particles as determined by optical densitometry (more preferably 10¹¹-10¹³ viral particles, most preferably 10¹² viral particles). This type of injection enables local transfection of a desired number of cells, in the affected myocardium with function-enhancing or phenotype-determinant peptide-encoding genes, thereby maximizing therapeutic efficacy of gene transfer, and minimizing undesirable effects at extracardiac sites and the possibility of an inflammatory response to viral proteins. A tissue- or cell-specific promoter may be used, for example, to securely enable expression limited to the cardiac myocytes so as to avoid the potentially harmful effects in non-cardiac tissues. Thus delivery of the transgenes in this matter may result in targeted gene expression.

Any variety of coronary catheter, or a Stack perfusion catheter, for example, can be used in the present invention.

Vectors comprising transgenes for use in the present invention can also be introduced directly into the heart muscle, for example by direct injection during a surgical procedure, or by using special catheters which can be inserted into a peripheral blood vessel and guided into the ventricle of the heart to inject the vector directly into the wall of the heart. Other devices such as balloon catheters may be capable of delivering vectors to the walls of an artery. While potentially applicable, such procedures are presently less preferred than methods for intravascular delivery to the myocardium as described by Hammond et al. in U.S. Pat. No. 5,792,453, issued Aug. 11, 1998.

Therapeutic Applications:

The vectors of the present invention allow for highly efficient gene transfer in vivo without cytopathic effect or inflammation in the areas of gene expression.

In cases of myocardial infarction, treatment during the healing phase can result in muscle regeneration. In the treatment of congestive heart failure, gene therapy can be prophylactic after a first myocardial infarction, or after the first clinical sign of heart failure. Application of these techniques clinically will be of great utility, especially initially in those with inoperative coronary artery disease, myocardial infarction, cardiomyopathy or heart failure of any cause.

Compositions or products of the invention may conveniently be provided in the form of formulations suitable for intracoronary administration. A suitable administration format may best be determined by a medical practitioner for each patient individually. Suitable pharmaceutically acceptable carriers and their formulation are described in standard formulations treatises, e.g., Remington's Pharmaceuticals Sciences by E. W. Martin. See also Wang, Y. J. and Hanson, M. A., “Parental Formulations of Proteins and Peptides: Stability and Stabilizers”, Journals of Parental Sciences and Technology, Technical Report No. 10, Supp. 42:2S (1988). Vectors of the present invention should preferably be formulated in solution at neutral pH, for example, about pH 6.5 to about pH 8.5, more preferably from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, more preferably from 0.15% to 0.4% metacresol. Sucrose is preferred for stabilizing the adenovirus and prolonging storage time without loss of potency. The desired isotonicity may be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is preferred particularly for buffers containing sodium ions. If desired, solutions of the above compositions may also be prepared to enhance shelf life and stability. The therapeutically useful compositions of the invention are prepared by mixing the ingredients following generally accepted procedures. For example, the selected components may be mixed to produce a concentrated mixture which may then be adjusted to the final concentration and viscosity by the addition of water and/or a buffer to control pH or an additional solute to control tonicity.

For use by the physician, the compositions will be provided in dosage form containing an amount of a vector of the invention which will be effective in one or multiple doses to induce the desired level of protein expression. As will be recognized by those in the field, an effective amount of therapeutic agent will vary with many factors including the age and weight of the patient, the patient's physical condition, and other factors.

The virus is preferably preserved in a sucrose phosphate buffered saline solution, well known to those skilled in the art. As noted, the exact dose to be administered is determined by the attending clinician, but is typically in 1 ml phosphate buffered saline and sucrose. The virus may be diluted prior to administration in phosphate buffered saline. The therapeutic techniques of the present invention can also be combined with other compositions and/or methods that are used in the treatment of patients suffering from these heart diseases, as will be appreciated by medical practitioners and others of skill in the art. By way of illustration, the treatments may be performed in conjunction with the use of angiotensin converting enzyme (ACE) inhibitors or other pharmacological agents known to improve patients' conditions and/or symptoms.

To assist in understanding the present invention, the following Examples are provided. The experiments relating to this invention should not, of course, be construed as specifically limiting the invention and such variations of the invention, now known or later developed, which would be within the purview of one skilled in the art are considered to fall within the scope of the invention as described herein and hereinafter claimed.

### EXAMPLES

The preferred methods of the present invention relate to the enhancement of cardiac function in patients, preferably through regeneration of cardiomyocytes after myocardial infarction or through local production of hormones that improve cardiac function. Examples of a porcine model used for delivery of transgenes to the heart are presented in PCT Publication Number WO96/26742; WO98/10085, WO98/50079, and U.S. Pat. No. 5,792,493 issued Aug. 11, 1998; all hereby incorporated by reference herein.

### Example 1

### Treatment of Myocardial Infarction

The formation of collagen-rich scar tissue by granulation after acute myocardial infarction has a negative effect on cardiac function in three ways: 1) muscle is replaced by noncontractile scar; 2) scar can expand during cardiac contraction, causing lost work for the contractile muscle; and 3) it increases the size and effective radius of the ventricle, which increases wall stress and places excessive burden on residual muscle.

By converting granulation tissue into cardiac muscle, the infarction will heal by replacing the dead tissue with normal, contractile, muscle cells.

The transfer of certain genes to heart cells will help to regenerate heart muscle to mitigate the effect of muscle necrosis and improve heart performance following a heart attack.

Investigators using model systems in flies have found that deletion of a gene known as “Tinman” results in the failure to form a cardiac tube (Bodmer, R. Development 118:719-728, (1993)). A human homolog of Tinman is NKX-2.5. NKX-2.5 has been found to be expressed in cells involved in commitment to and/or differentiation of the myocardial lineage. Lints, T. J. et al., Development 119:419-431 (1993). The sequence of NKX-2.5 has also been determined. Id.; corrected at Lints et al., Development 119:969 (1993). NKX-2.5 has also been referred to as “Csx” by Komuro and Izumu, Proc. Nat'l. Acad. Sci. 90:8145-8149 (1993). NKX-2.5 (also sometimes written as Nkx2-5), and other cardiac related genes are reviewed in Bodmer, R. et al., Dev. Gen., 22:181-186 (1998). Other members of the related NKX family in vertebrates which appear to be involved in cardiogenesis, also share (with NKX-2.5) conserved sequence domains including a homeobox domain, a TN domain and an NK2 domain. See, e.g., Bodmer, R. et al., Dev. Gen., 22:181-186 (1998), and Newman et al., Dev. Gen. 22:230-238 (1998). Other genes downstream from NKX-2.5 include GATA4, and MEF2, a myocyte-specific enhancer binding factor (Gossett, L. A., et al., Mol. Cell. Biol. 9:5022-33 (1989); Parmacek, M. S. et al., Mol. Cell. Biol., 14:1870-85 (1994); Cheng, T. C., et al., Science 261:215-218 (1993); Martin, J. F., et al., Proc. Natl. Acad. Sci. USA 90:5282-5286 (1993); Edmondson, D. G. et al., Mol. Cell. Biol. 12:3665-77 (1992)). Further information regarding genes involved in cardiac differentiation can be found in the literature regarding heart development. See, e.g., Mably, J., et al., Circ. Res. (1996) 79:4-13; Mohun, T., et al. Curr. Opin. Gen. & Dev. (1997) 7:628-633; Jiang, Y., et al., Dev. Gen. (1998) 22:263-277; Newman, C., et al., Dev. Gen. (1998) 22:230-238; Durocher, D., et al., Dev. Gen. (1998) 22:250-262; Bodmer, R., et al., Dev. Gen. (1998) 22:181-186; Tanaka, M., et al., Dev. Gen. (1998) 22:239-249; and Patterson, K., et al., Current Topics in Developmental Biology (1998) 40:1-44. These references are hereby incorporated by reference herein.

A coronary artery that is occluded may be opened by any convenient means such as thrombolysis, angioplasty, or atherectomy. A recombinant viral vector, containing a NKX-2.5, MEF2, GATA4 and/or other cardiomyocyte differentiating peptide or cardiac myocyte lineage-specifying gene is injected into the coronary artery, preferably within about 2 days to 2 weeks following myocardial infarction.

In another preferred method, the administration of a vector containing a human growth hormone gene may be beneficial.

The high first-pass uptake of virus within the heart prevents significant gene transfer to other organs and unwanted ectopic production of cardiomyocytes. Transient availability of the transgene can be desirable. For example, once a fibroblast has changed phenotype into a cardiomyocyte, the change is self sustaining. Thus necrotic tissue can be replaced by new cardiomyocytes.

Candidate genes for this method such as those described above may be screened by use of recombinant viral vectors in cell culture experiments to test for transdifferentiation of non-cardiac cells. Cell types to be tested preferably include, but are not limited to, adult and neonatal cardiac fibroblasts, embryonic stem cells, P19 embryonal carcinoma cells, skeletal myoblasts, bone marrow stromal cells, and smooth muscle cells. Atrial natriuretic factor, myosin light chain 2v, α-myosin heavy chain, and β-myosin heavy chain can be used as indicators of cardiac differentiation. In addition, the effects of the delivery of these genes may be tested in vivo in a rat cardiac injury model or other suitable animal model.

By way of illustration, virus containing the transgene is grown to high titer by methods known to those skilled in the art. To deliver the vector, a cardiac catheterization can be performed by any standard, well known method. The catheter tip is preferably placed deep within the coronary artery lumen, or more preferably, in any sequence, within the lumen of each of the three major coronary arteries sequentially, and/or any by pass grafts such as vein bypass grafts or internal mammary grafts. The gene therapy vector is injected separately into each major vessel or coronary artery branch.

### Example 2

### Gene-Transfer-Mediated Therapy for Heart Muscle Cell Regeneration Using NKX-2.5

Although this Example describes the delivery of the NKX-2.5 gene to heart cells, those of ordinary skill in the art will understand that other cardiac function enhancing peptides, such as MEF2 and GATA4, or other cardiomyocyte differentiating factors may be used following the methods described herein without undue experimentation. DNA encoding NKX-2.5 is used for the regeneration of heart muscle through gene delivery to the heart as described, for example, in International Patent Application No. PCT/US96/02631, published Sep. 6, 1996, as WO96/26742, hereby incorporated by reference herein in its entirety.

Adenoviral Constructs

A helper independent replication deficient human adenovirus 5 system may be used for gene-transfer. By way of illustration, a nucleic acid molecule coding for a cardiac enhancing peptide may be cloned into the polylinker of plasmid ACCMVPLPA which contains the CMV promoter and SV40 polyadenylation signal flanked by partial adenoviral sequences from which the E1A and E1B genes (essential for viral replication) have been deleted. This plasmid is co-transferred (lipofection) into 293 cells with plasmid JM17 which contains the entire human adenoviral 5 genome with an additional 4.3 kb insert making pJM17 too large to be encapsulated. Homologous rescue recombination results in adenoviral vectors containing the transgene in the absence of E1A and E1B sequences. Although these recombinants are nonreplicative in mammalian cells, they can propagate in 293 cells which have been transformed with E1A and E1B and provided these essential gene products in trans. Transfected cells are monitored for evidence of cytopathic effect which usually occurs 10-14 days after transfection. To identify successful recombinants, cell supernatant from plates showing a cytopathic effect is treated with proteinase K (50 mg/ml with 0.5% sodium dodecyl sulfate and 20 mM EDTA) at 56° C. for 60 minutes, phenol/chloroform extracted and ethanol precipitated. Successful recombinants are then identified with PCR using primers (Biotechniques, 15:868-72, 1993) complementary to the CMV promoter and SV40 polyadenylation sequences to amplify the NKX-2.5 nucleic acid insert and primers (Biotechniques, 15:868-72, 1993) designed to concomitantly amplify adenoviral sequences. Successful recombinants then are plaque purified twice. Viral stocks are propagated in 293 cells to titers ranging between 10¹⁰ and 10¹² viral particles, and are purified by double CsCl gradient centrifugation, or by column chromatography or other suitable means, prior to use. The system used to generate recombinant adenoviruses imposed a packing limit of 5 kb for transgene inserts. The NKX-2.5 gene driven by the CMV promoter and with the SV40 polyadenylation sequences is well within the packaging constraints. Recombinant vectors are plaque purified by standard procedures. The resulting viral vectors are propagated on 293 cells to titers in the 10¹⁰-10¹² viral particles range. Cells are infected at 80% confluence and harvested at 36-48 hours. After freeze-thaw cycles the cellular debris is pelleted by standard centrifugation and the virus further purified by double CsCl gradient ultracentrifugation (discontinuous 1.33/1.45 CsCl gradient: cesium prepared in 5 mM Tris, 1 mM EDTA (pH 7.8); 90,000×g (2 hr), 105,000×g (18 hr)). Prior to in vivo injection, the viral stocks are desalted by gel filtration through Sepharose columns such as G25 Sephadex. Alternate column chromatography methods providing advantages in purity can also be used. The resulting viral stock has a final viral titer approximately in the 10¹⁰-10¹² viral particles range. The adenoviral construct should thus be highly purified, with few or no wild-type (potentially replicative) virus.

Canine Myocardial Infarction Model

A canine model of myocardial infarction may be used to determine the level of heart muscle regeneration after delivery of transgene such as NKX-2.5. (Pouleur, M., et al. Europ. J. Clin. Invest. 13:331-8 (1983)). A survival thoractomy is performed on a dog using methods known to those of ordinary skill in the art. A hydraulic cuff occluder is inserted on the right coronary artery. Ten days later, an infarction is induced using a syringe of water which is inserted into the tubing of the occluder. After infarction occurs, after about 8 hours, the cuff occluder is removed. From three to fourteen days later, the adenovirus—NKX-2.5 vector is delivered. Injection of the construct (4.0 ml containing about 10¹¹ viral particles of adenovirus) is performed by injecting 2.0 ml into the coronary artery. Animals are anesthetized, and arterial access acquired via the right carotid by cut-down; a 5F Cordis sheath is then placed. A 5F Multipurpose (A2) coronary catheter is used to engage the coronary arteries. The catheter tip is then placed 1-3 cm within the arterial lumen so that minimal material is lost to the proximal aorta during injection.

Once gene transfer is performed, three strategies are used to establish successful incorporation and expression of the gene: (1) Some constructs may include a reporter gene (lacZ); (2) myocardium from the relevant beds is sampled, and immunoblotting is performed to quantitate the presence of NKX-2.5 protein; and (3) PCR is used to detect NKX-2.5 mRNA and DNA.

The helper independent replication deficient human adenovirus 5 system is used to prepare transgene containing vectors. The material injected in vivo should be highly purified and contain no wild-type (replication competent) adenovirus. Thus adenoviral infection and inflammatory infiltration in the heart are minimized. By injecting the material directly into the lumen of the coronary artery by coronary catheters, it is possible to target the gene effectively. When delivered in this manner there should be no transgene expression in hepatocytes, and viral RNA should not be found in the urine at any time after intracoronary injection.

At completion of the study, animals are sacrificed and the infarct is removed. The size of the infarct is compared in control (no vector, or adenovirus vector without transgene) animals and animals that received the NKX-2.5 gene. The infarcts are also examined for evidence of newly regenerated cardiac myocytes using methods known to those of skill in the art. The infarct size will be larger in control animals than those that received the NKX-2.5 gene. The animals that received the NKX-2.5 gene will also have more newly regenerated cardiac myocytes than the control animals.

Basal function or wall thickening may also be measured before and after delivery of the transgene. Animals that receive the transgene will have greater wall thickness due to newly regenerated cardiomyocytes.

Echocardiographic measurements are made using standardized criteria (Sahn, et al., Circulation 58:1072, 1978). End-diastolic wall thickness (EDWTh) and end-systolic wall thickness (ESWTh) are measured from 5 continuous beats and averaged. Percent wall thickening (%WTh) is calculated [(EDWTh-ESWTh)/EDWTh]×100. Data should be analyzed without knowledge of which gene the animals had received. To demonstrate reproducibility of echocardiographic measurements, animals should be imaged on two consecutive days, showing high correlation (r²=0.90; p=0.005).

To establish that muscle cell regeneration results from transgene expression, PCR and RT-PCR may be used to detect transgenic NKX-2.5 DNA and mRNA in myocardium from animals that have received the NKX-2.5 gene transfer. Using a sense primer to the CMV promoter [GCAGAGCTCGTTTAGTGAAC] and antisense primer to the internal NKX-2.5 gene sequence PCR is used to amplify the expected 500 bp fragment. Using a sense primer to the beginning of the NKX-2.5 protein sequence and an antisense primer to the internal NKX-2.5 protein gene sequence RT-PCR is used to amplify the expected 400 bp fragment.

Finally, using an antibody directed against NKX-2.5 protein expression may be demonstrated 48 hours as well as 14±1 days after gene transfer in cells and myocardium from animals that have received gene transfer with an NKX-2.5 gene.

### Example 3

### Treatment of CHF with Directed Delivery of Human Growth Hormone

Patients with congestive heart failure may be treated with human growth hormone (HGH) to help to alleviate the progressive dilation of the heart and worsening of heart function. Human growth hormone has systemic effects that may be undesirable.

If the effects of growth hormone could be confined to the heart, such systemic side effects could be avoided. This embodiment uses human growth hormone cDNA in a viral vector delivered by intracoronary injection. High first pass uptake of the vector concentrates gene transfection to the heart. Local production of the growth factor limits circulation and avoids systemic side effects. Those of ordinary skill in the art will understand that the dose of the vector is calibrated to ensure adequate myocardial levels for the treatment of heart failure, but insufficient to cause significant circulating levels and systemic effects.

In an additional embodiment, a chimeric protein is generated by adding the coding sequence for the proteoglycan binding domain of human VEGF₁₄₅, PCT Publication WO98/10071, published Mar. 12, 1998, hereby incorporated by reference herein; Charnock and Jones et al., Biology of Reproduction 48:1120-28 (1993); Gitay-Goren et al., J. Biol. Chem., 271:5519-23 1996)) to the 5′ end of the HGH, cDNA. In other embodiments, the proteoglycan binding domain of other tightly bound proteins can be used. In yet another embodiment the DNA sequence is preceded by the alpha myosin heavy chain promoter to enhance cardiac specific effects.

The replication incompetent adenovirus containing the transgene is grown to high titer by methods known to those skilled in the art. To deliver the vector, a cardiac catheterization is performed by any standard, well known method.

In gene delivery methods of the present invention, preferably the catheter tip is placed deep within the coronary artery lumen, or preferably, in any sequence, within the lumen of each of the three major coronary arteries sequentially. The gene therapy vector is injected separately into each major coronary artery, or coronary artery branch.

In another embodiment, a vector could be injected systemically, but cardiac-specific expression obtained by using a cardiac specific promoter. For example, such a promoter might be the alpha-myosin heavy chain promoter to give cardiomyocyte specific expression.

### Example 4

### Treatment of CHF by BCL-2 Gene Therapy—Prevention of Apoptosis

Chronic CHF is associated with the progressive loss of cardiomyocytes by apoptosis.

BCL-2 displayed on the mitochondria, is an important regulator of apoptosis. A viral vector containing the cDNA for BCL-2 is injected intracoronary into all three major coronary arteries. The forced over-expression of BCL-2 in cardiomyocytes increases their refractoriness to apoptosis, prevents cell loss in the failing heart, and halts the progression of CHF. See, Kaistura, J., et al., Exp. Cell Res. 219:110-121 (1995). Hockenbery, D. M., et al., Cell, 75:241-51 (1993); Misao, J. et al., Circulation 94:1506-1512 (1996); Kirshenbaum, L. A., and deMoissac, D., Circulation, 96:1580-85 (1997).

The replication-incompetent adenovirus containing the transgene is grown to high titer by methods known to those skilled in the art. To deliver the vector, a cardiac catheterization is performed by any standard, well known method. The catheter tip is placed deep within the coronary artery lumen, or preferably, in any sequence, within the lumen of each of the three major coronary arteries sequentially. The gene therapy vector is injected separately into each major coronary artery, or coronary artery branch.

In another embodiment, a vector could be injected systemically, but cardiac specific expression obtained by using a cardiac specific promoter. For example, such a promoter might be the alpha-myosin heavy chain promoter to give cardiomyocyte specific expression.

There are a number of other anti-apoptotic genes in the BCL-2 family, and those skilled in the art will recognize that these genes would be useful using the methods described herein. In addition, in another preferred method, a vector containing a human growth hormone gene may be administered.

### Example 5

### Porcine Model of Congestive Heart Failure

The methods disclosed herein relating to the construction and propagation of the adenoviral constructs and other vectors may be used for this example.

Other examples of a porcine model used for adenoviral delivery of transgenes are presented in PCT WO98/10085, published Mar. 12, 1998, and WO98/50079, published Nov. 12, 1998, hereby incorporated by reference herein.

Animals and Surgical Procedure

Yorkshire pigs (Sus scrofa) weighing 40±6 kg are anesthetized with ketamine (50 mg/kg IM) and atropine sulfate (0.1 mg/kg IM) followed by sodium amytal (100 mg/kg IV). After endotracheal intubation, halothane (0.5% to 1.5%) is delivered by a pressure-cycled ventilator throughout the procedure. At left thoracotomy, catheters are placed in the aorta, pulmonary artery, and left atrium. A Konigsberg micromanometer is placed into the left ventricular apex, and an epicardial unipolar lead is placed 1.0 cm below the atrioventricular groove in the lateral wall of the left ventricle. The power generator (Spectrax 5985; Medtronic, Inc.) is inserted into a subcutaneous pocket in the abdomen. One group of animals is instrumented with a flow probe (Transonic, Inc.) around the main pulmonary artery. The pericardium is loosely approximated and the chest closed. Seven to 10 days after thoracotomy, baseline measures of hemodynamics, left ventricular function, and myocardial blood flow are made. Ventricular pacing is initiated (220±9 bpm for 26±4 days). The stimulus amplitude is 2.5 V, the pulse duration 0.5 ms. Additional pigs (40±7 kg) are used as controls; One group of these control animals undergo thoracotomy and instrumentation without pacing and are killed 30±7 days after initial thoracotomy.

Hemodynamic Studies:

Hemodynamic data are obtained from conscious, unsedated animals after the pacemaker has been inactivated for at least 1 hour and animals are in a basal state. All data are obtained in each animal at 7-day intervals. Pressures are obtained from the left atrium, pulmonary artery, and aorta. Left ventricular dP/dt is obtained from the high-fidelity left ventricular pressure. Pulmonary artery flow is recorded. Aortic and pulmonary blood samples are obtained for calculation of arteriovenous oxygen content difference.

Echocardiographic Studies:

Two-dimensional and M-mode images are obtained with a Hewlett Packard Sonos 1500 imaging system. Images are obtained from a right parasternal approach at the mid-papillary muscle level and recorded on VHS tape. Measurements are made according to criteria of the American Society of Echocardiography (Sahn, D J, et al., Circulation. 58:1072-1083 (1978)). Because of the midline orientation of the porcine interventricular septum (IVS) and use of the right parasternal view, short-axis M-mode measures are made through the IVS and the anatomic lateral wall. All parameters, including end-diastolic dimension (EDD), end-systolic dimension (ESD), and wall thickness, are measured on at least five random end-expiratory beats and averaged. End-diastolic dimension is obtained at the onset of the QRS complex. End-systolic dimension is taken at the instant of maximum lateral position of the IVS or at the end of the T wave. Left ventricular systolic function is assessed by use of fractional shortening, RS=[(EDD−ESD)EDD]×100. Percent wall thickening (%WTh) is calculated as %WTh=[(ESWTh-EDWTh)/EDWTh]×100. To demonstrate reproducibility of echocardiographic measurements, animals were imaged on 2 consecutive days before the pacing protocol was initiated. All of these measurements are obtained with pacemakers inactivated.

Myocardial Blood Flow:

Myocardial blood flow is determined by the radioactive microsphere technique as described in detail in previous reports. (Roth, D M, et al., Am. J. Physiol. 253:H1279-H1288 (1987); Roth, D M, et al., Circulation 82:1778-1789 (1990)) Transmural samples from the left ventricular lateral wall and IVS are divided into endocardial, midwall, and epicardial thirds, and blood flow to each third and transmural flow is determined. Transmural sections are taken from regions in which echocardiographic measures have been made so that blood flow and functional measurements correspond within each bed. Microspheres are injected in the control state (unpaced), at the initiation of ventribular pacing (225 bpm), and then at 7-day intervals during ventricular pacing at 225 bpm: microspheres are also injected with the pacemakers inactivated at 14 days (n=4) and 21 to 28 days (n=3). By dividing myocardial blood flow by the heart rate (recorded during microsphere injection), the myocardial blood flow per beat is calculated (Indolfi, C., et al., Circulation 80:933-993 (1989)). Mean left atrial and mean arterial pressures are recorded during microsphere injection so that an estimate of coronary vascular resistance may be calculated; coronary vascular resistance index (min Hg≅mL⁻¹≅min⁻¹≅g⁻¹)=mean arterial pressure−mean left atrial pressure) transmural coronary blood flow.

Systolic Wall Stress:

Meridional end-systolic wall stress is calculated (Riechek, N., et al., Circulation 65:99-108 (1982)) using the equation meridional end-systolic wall stress (dynes)=(0.334×P×D)÷[h(I−h/D)], where P is left ventricular end-systolic pressure in dynes, D is left ventricular end-systolic diameter in cm, and h is end-systolic wall thickness. Meridional end-systolic wall stress is calculated for both lateral wall and IVS before the initiation of pacing and subsequently at weekly intervals (pacemaker off).

Terminal Surgery:

After 26±2 days of pacing, animals are anesthetized and incubated, and midline sternotomies are made. The still-beating hearts are submerged in saline (4° C.), the coronary arteries are rapidly perfused with saline (4° C.), the right ventricle and left ventricle (including IVS) are weighed, and transmural samples from each region are rapidly frozen in liquid nitrogen and stored at a temperature of −70° C.

Adenine Nucleotides:

ATP and ADP are measured in transmural samples of the IVS and lateral wall in animals with heart failure (paced 28 days) and control animals. The samples from the animals with heart failure are obtained with the pacemakers off (60 minutes) on the day the animals were killed. Samples are obtained identically in all animals. ATP and ADP are measured in a Waters high-performance liquid chromatograph as previously described. (Pilz, R. B., et al., J. Biol. Chem. 259:2927-2935 (1984))

Statistical Analysis:

Data are expressed as mean±SD. Specific measurements obtained in the control (prepaced) state and at 1-week intervals during pacing are compared by repeated measures ANOVA (Crunch4, Crunch Software Corp.). In some comparisons (lateral wall versus IVS, for example), two-way ANOVA is used. Post hoc comparisons are performed with the Tukey method.

ANOVA is conducted on animals at four time points: control (prepacing), 7 days, 14 days, and 21 to 28 days. The null hypothesis is rejected when P<0.05 (two-tailed).

Global Left Ventricular Function:

Left ventricular function is assessed by echocardiography and hemodynamic variables after pacemakers have been inactivated.

Left Ventricular Regional Function:

With the pacemaker inactivated, regional left ventricular function is assessed by measurement of percent wall thickening of the left ventricular lateral wall and IVS.

The effect of the BCL-2 or HGH gene in a model of congestive heart failure is then assessed. After delivery of the transgene according to methods presented in this application, and to those known to those of skill in the art, control and test animals are assessed for signs of relief from congestive heart failure using methods known to those of skill in the art. Such tests include: testing for fractional shortening in echocardiography, hemodynamic measurements including cardiac output and contactility, and tests of global ventricular function.

Utilization of this model for growth hormone gene transformation can employ intracoronary injection or coronary sinus retroperfusion as described above. Essentially identical or similar measurements are readily made in the dog micro-embolyzation model of congestive heart failure to demonstrate efficacy, for example. (Hori, M., et al., Am. J. Physiol. 267:H1483-95 (1994); Todaka, K., Am. J. Physiol. 272:H186-94 (1997)). A dog congestive heart failure model may also be used, as known by those of ordinary skill in the art. (Engler, R., et al., in Clinical and Experimental Hypertension, Part A, Theory and Practice 4:639-59 (1982); Pouleur, H., et al., Europ. J. Clin. Invest. 13:331-8 (1983)).

### Example 6

### Prevention of Transplant Rejection and Delay of Atherosclerosis

Gene transfer to induce display of Fas ligand on the plasma membrane of endothelial cells and cardiomyocytes in transplanted organs will induce death in immune competent T cells that are directed at a transplanted organ. Eventually T cells directed at transplant (foreign) antigens will become depleted and immune tolerance will be induced. In addition, transplanted hearts are subject to accelerated atherosclerosis. An important mechanism of this major complication is mediated by immune injury to the coronary vessels which display foreign antigens. The display of the Fas ligand on the endothelial cells and vascular smooth muscle cells of the coronary arteries will delay the development of atherosclerosis.

Thus, in the present example, the gene coding for the Fas ligand is inserted into a viral vector, such as the adenoviral or adeno-associated viral vectors described herein. Such vectors are administered to the transplanted organ either before, during, or after the organ transplant to induce expression of the Fas ligand on the transplanted organ. The vector is used to transfect the transplant organ in vitro, before the transplant, so that the transplanted tissue expresses the Fas ligand.

To deliver the vector before transplant, the transplant heart undergoes extracorporeal perfusion and the gene therapy vector is circulated in the perfusate.

Although preferred embodiments are specifically described herein, it will be appreciated that many modifications and variations of the present invention are possible in light of the above teachings and within the purview of the appended claims without departing from the spirit and intended scope of the invention.

1. A method for improving or maintaining cardiac function in a patient comprising delivering a vector to the heart of said patient, said vector comprising a transgene coding for a cardiomyocyte-differentiating peptide.

2. A method for improving or maintaining cardiac function in a patient comprising delivering a vector to the heart of said patient, said vector comprising a transgene coding for a peptide selected from the group consisting of NKX-2.5, MEF2, and GATA4.

3. A method for stimulating heart muscle regeneration in a patient comprising delivering a vector comprising a transgene to the heart of said patient, wherein said transgene encodes a cardiomyocyte-differentiating peptide.

4. The method of claim 3 wherein said cardiomyocyte-differentiating peptide is selected from the group consisting of NKX-2.5, MEF2, and GATA4.

5. A method for improving or maintaining cardiac function in a patient comprising delivering a vector to the heart of said patient, said vector comprising a transgene coding for an anti-apoptotic protein.

6. The method of claim 5, wherein said anti-apoptotic protein is a BCL-2-family apoptotic protein.

7. A method for improving or maintaining cardiac function in a patient comprising delivering a vector to the heart of said patient, said vector comprising a transgene coding for BCL-2.

8. A method for treating congestive heart failure in a patient comprising delivering a vector comprising a transgene coding for BCL-2 to the heart of said patient.

9. The method of claim 8 wherein said treatment prevents cardiac cell loss.

10. The method of claim 8 wherein said treatment renders the cardiomyocytes more resistant to apoptosis.

11. A method for improving or maintaining cardiac function in a patient comprising delivering a vector comprising a transgene coding for HGH to the heart of said patient.

12. A method for treating congestive heart failure in a patient comprising delivering a vector comprising a transgene coding for HGH to the heart of said patient.

13. The method of claim 11 or 12, wherein said HGH transgene is fused at its 5′ end to a proteoglycan binding domain of VEGF₁₄₅.

14. The method of any of claims 1-4 or 11-13, wherein said delivery is after a myocardial infarction.

15. The method of any of claims 1-14, wherein the vector is delivered to cardiac myocytes.

16. The method of any of claims 1-4, wherein the vector is delivered to myofibroblasts.

17. The method of any of claims 1-14, wherein said transgene is a human gene.

18. The method of any of claims 1-17 wherein said vector is delivered by coronary sinus retroprofusion.

19. The method of any of claims 1-14, wherein the vector is delivered by intracoronary injection into one or both coronary arteries.

20. The method of any of claims 1-14, wherein the vector is delivered to a blood vessel supplying blood to the myocardium of the heart, wherein said blood vessel is selected from the group consisting of a coronary artery, a saphenous vein graft, and an internal mammary artery graft.

21. The method of any of claims 1-14 wherein said vector is a replication-deficient adenovirus vector.

22. The method of claim 21 wherein said replication-deficient adenovirus vector is delivered to the myocardium of the patient by intracoronary injection directly into one or both coronary arteries, said vector comprising a transgene coding for a peptide selected from the group consisting of NKX-2.5, MEF2, GATA4, BCL-2, and HGH, and capable of expressing the transgene in the myocardium.

23. The method of any of claims 1-14, wherein the patient is human.

24. The method of any of claims 1-14, wherein the patient has congestive heart failure.

25. The method of claim 22, wherein a single injection of said vector is delivered.

26. The method of claim 22, wherein about 10¹⁰ to about 10¹⁴ adenovirus vector particles are delivered in the injection.

27. The method of claim 22, wherein about 10¹¹ to about 10¹³ adenovirus vector particles are delivered in the injection.

28. The method of claim 22, wherein about 10¹² adenovirus vector particles are delivered in the injection.

29. The method of claim 22, wherein said transgene is driven by a CMV promoter which is contained in the vector.

30. The method of claim 22, wherein said transgene is driven by a heart cell-specific promoter which is contained in the vector.

31. The method of claim 30, wherein said heart cell-specific promoter has the sequence of ventricular myosin light chain-2 promoter or alpha myosin heavy chain promoter.

32. The method of claim 22, wherein said transgene is driven by a fibroblast-specific promoter which is contained in the vector.

33. The method of claim 22, wherein said transgene is driven by a myofibroblast-specific promoter which is contained in the vector.

34. The method of claim 22, wherein said intracoronary injection is conducted about 1-3 cm into the lumens of the left and right coronary arteries.

35. The method of claim 22, wherein said intracoronary injection is conducted about 1-3 cm into the lumens of a saphenous vein graft and/or an internal mammary artery graft in addition to coronary artery.

36. The method of claim 22, wherein said replication-deficient adenovirus vector comprises a partial adenoviral sequence from which the E1A and E1B genes have been deleted.

37. A kit for intracoronary injection of a recombinant vector expressing a cardiomyocyte-differentiating peptide comprising:
a nucleic acid molecule encoding a cardiomyocyte-differentiating peptide cloned into a vector suitable for expression of said polynucleotide in a heart cell,
a suitable container for said vector, and
instructions for injecting said vector into a patient.

38. A kit for intracoronary injection of a recombinant vector expressing a peptide selected from the group consisting of NKX-2.5, MEF2, GATA4, BCL-2, HGH, and Fas ligand comprising:
a nucleic acid molecule encoding NKX-2.5, MEF2, GATA4, BCL-2, HGH, or Fas ligand cloned into a vector suitable for expression of said polynucleotide in a heart cell,
a suitable container for said vector, and
instructions for injecting said vector into a patient.

39. The kit according to claim 37 or 38, wherein said nucleic acid molecule is cloned into an adenovirus expression vector.

40. The method according to any of claims 1-14, wherein an inflatable balloon catheter coated with said vector is employed to deliver said transgene.

41. A filtered injectable adenovirus vector preparation, comprising: a recombinant adenoviral vector, said vector containing no wild-type virus and comprising:
a partial adenoviral sequence from which the E1A/E1B genes have been deleted,
and a transgene coding for a NKX-2.5, MEF2, GATA4, BCL-2, HGH, or Fas ligand driven by a promoter flanked by the partial adenoviral sequence; and
a pharmaceutically acceptable carrier.

42. A kit for coronary sinus retroprofusion of a recombinant vector expressing a peptide selected from the group consisting of NKX-2.5, MEF2, GATA4, BCL-2, HGH, and Fas ligand comprising:
a nucleic acid molecule encoding NKX-2.5, MEF2, GATA4, BCL-2, HGH, or Fas ligand cloned into a vector suitable for expression of said polynucleotide in a heart cell,
a suitable container for said vector, and
instructions for injecting said vector into a patient.

43. A method of reducing the likelihood of rejection of a transplanted organ in a patient comprising delivering a vector to the transplanted organ, said vector comprising a transgene coding for a Fas ligand.

44. The method of claim 43, wherein said transplanted organ is heart tissue.

45. The method of claim 43, wherein said vector is delivered to the transplanted organ before transplanting the organ into a patient.

46. The method of claim 43, wherein said vector is delivered to the transplanted organ after transplanting the organ into a patient.

47. The method of claim 43, wherein said patient is human.

48. The method of claim 44 wherein the vector is delivered by coronary sinus retroprofusion.

49. The method of claim 44, wherein the vector is delivered by intracoronary injection into one or both coronary arteries.

50. The method of claim 44, wherein the vector is delivered to a blood vessel supplying blood to the myocardium of the heart, wherein said blood vessel is selected from the group consisting of a coronary artery, a saphenous vein graft, and an internal mammary artery graft.

51. The method of claim 44, wherein said vector is a replication-deficient adenovirus vector.

52. The method of claim 51, wherein said replication-deficient adenovirus vector is delivered to the myocardium of the patient by intracoronary injection directly into one or both coronary arteries, said vector comprising a transgene coding for Fas ligand, and capable of expressing the transgene in the myocardium.

53. The method of claims 44, wherein the patient is human.

54. The method of claims 44, wherein the patient has congestive heart failure.

55. The method of claim 49, wherein a single injection of said vector is delivered.

56. The method of claim 49, wherein about 10¹⁰ to about 10¹⁴ adenovirus vector particles are delivered in the injection.

57. The method of claim 49, wherein about 10¹¹ to about 10¹³ adenovirus vector particles are delivered in the injection.

58. The method of claim 49, wherein about 10¹² adenovirus vector particles are delivered in the injection.

59. The method of claim 44, wherein said transgene is driven by a CMV promoter which is contained in the vector.

60. The method of claim 44, wherein said transgene is driven by a heart cell-specific promoter which is contained in the vector.

61. The method of claim 60, wherein said heart cell-specific promoter has the sequence of ventricular myosin light chain-2 promoter or alpha myosin heavy chain promoter.

62. The method of claim 49, wherein said intracoronary injection is conducted about 1-3 cm into the lumens of the left and right coronary arteries.

63. The method of claim 49, wherein said intracoronary injection is conducted about 1-3 cm into the lumens of a saphenous vein graft and/or an internal mammary artery graft in addition to coronary artery.

64. The method of claim 49 wherein said replication-deficient adenovirus vector comprises a partial adenoviral sequence from which the E1A and E1B genes have been deleted.

65. The method according to claim 44, wherein an inflatable balloon catheter coated with said vector is employed to deliver said transgene.
